(19) United States
(12) Patent Application Publication
(10) Pub. No.: US 2002/0115625 A1
(43) Pub. Date: Aug. 22, 2002

(54) Immunization of infants

(76) Inventors: Adrian Bot, San Diego, CA (US); Constantin Bona, New York, NY (US)

Correspondence Address: BAKER BOTTS L.L.P., 44TH FLOOR, 30 ROCKEFELLER PLAZA, NEW YORK, NY, 10112-4498, US

(21) Appl. No.: 09/801,540

(22) Filed: Mar. 8, 2001

Related U.S. Application Data
(63) Continuation-in-part of application No. 09/308,511, filed on May 19, 1999.

(51) Int. Cl. A61K 48/00

(52) U.S. Cl. 514/44

(57) ABSTRACT

The present invention relates to methods and compositions which may be used to immunize infant mammals against a target antigen, wherein an immunogenically effective amount of a nucleic acid encoding a relevant epitope of a desired target antigen is administered to the infant. It is based, at least in part, on the discovery that such genetic immunization of infant mammals could give rise to effective cellular and humoral immune responses against target antigens.

### BACKGROUND OF THE INVENTION

A properly operating immune system enables an organism to maintain a healthy status quo by distinguishing between antigens associated with the organism itself, which are allowed to persist, and antigens associated with disease, which are disposed of. Decades ago, Burnet proposed that the immune system's ability to distinguish between “self” and “non-self” antigens results from the elimination of self-reactive lymphocytes in the developing organism (Burnet, 1959, The Clonal Selection Theory of Acquired Immunity, Vanderbilt Univ. Press, Nashville, Tenn.). The phenomenon wherein an organism loses the ability to produce an immune response toward an antigen is referred to as “tolerance”.

Over the years, a number of observations consistent with the clonal selection theory of tolerance have been documented. For example, genetically non-identical twin cattle, which share a placenta and are exposed to each other's blood cells in utero, fail to reject the allogeneic cells of their sibling as adults (Owen, 1945, Science 102:400). As another example, adult rodents that had been injected, at birth, with hemopoietic cells from a genetically distinct donor rodent strain were able to accept tissue transplants from that donor strain (Billingham et al., 1953, Nature 172:603; Billingham, 1956, Proc. R. Soc. London Ser. B. 239:44). However, in the early 1980's it was shown that the injection of minute amounts of antigen (namely an immunoglobulin expressing A48 regulatory idiotype) induced the expansion of helper T cells (Rubinstein et al., 1982, J. Exp. Med. 156:506-521).

The concept of tolerization is associated with the traditional belief that neonates are themselves incapable of mounting an effective immune response. It has been generally believed that neonates rely on maternal antibodies (passively transferred via the placenta) for immunity, until the neonate begins to synthesize its own IgG anti-bodies (at about 3-4 months after birth, in humans; Benjamini and Leskowitz, 1988, “Immunology, A Short Course”, Alan R. Liss, Inc., New York, p. 65).

More recently, several groups have reported findings that dispute the hypothesis that exposure to an antigen in early life disarms the ability of the immune system to react to that antigen.

Forsthuber et al. (1996, Science 271:1728-1730; “Forsthuber”) suggest that the impaired lymph node response of so-called “tolerized” mice was an artifact caused by a technical inability to assess immune function. They reported that neonatal mice, injected with hen egg lysozyme (HEL) in incomplete Freund's adjuvant (“IFA”) according to a protocol considered to induce tolerance in adults as well as neonates, displayed an impaired response in the lymph nodes consistent with tolerization. However, the spleen cells of these mice reportedly proliferated vigorously in response to HEL, a response previously unmeasurable due to technical limitations. The authors propose that neonatal injection did not tolerize, but rather induced functional memory cells that were detectable in spleen but not lymph nodes.

Sarzotti et al. (1996, Science 271:1726; “Sarzotti”) report that inoculation of newborn mice with a high dose of Cas-Br-M murine leukemia virus (“Cas”) does not result in immunological unresponsiveness, but rather leads to a nonproductive type 2 response which is likely to have a negative effect on the induction of mature effector cells. According to Sarzotti, clonal deletion of relevant CTL was not observed in mice infected at birth with a low dose of Cas.

Finally, Ridge et al. (1996, Science 271:1723-1726; “Ridge”) proposes that previous reports of tolerance induction may have been associated with a relative paucity of antigen presenting cells. Ridge observed the induction of CTL reactivity in neonatal mice injected with antigen expressed on dendritic cells (which are so-called professional antigen presenting cells).

The use of nucleic acids as vaccines was known prior to the present invention (see, for example, International Application Publication No. WO 94/21797, by Merck & Co. and Vical, Inc., and International Application Publication No. WO 90/11092). It was not known, however, that such vaccines could be used to induce an immune response in infant mammals.

### SUMMARY OF THE INVENTION

The present invention relates to methods and compositions which may be used to immunize infant mammals against a target antigen, wherein an immunogenically effective amount of nucleic acid encoding one or more relevant epitopes of one or more desired target antigens is administered to the infant. It is based, at least in part, on the discovery that such genetic immunization of infant mammals could give rise to effective cellular (including the induction of cytotoxic T lymphocytes) and humoral immune responses against target antigen. This ability to confer immunity to infants is surprising in the context of the conventional view, that exposure of an infant to an antigen induces tolerance rather than activation of the immune system. In addition, the ability of the present invention to induce a cellular immune response in infants is in contrast to the generally held concept that infants rely on maternal antibodies (rather than cellular elements) for immunity.

Moreover, the present invention may reduce the need for subsequent boost administrations (as are generally required for protein and killed pathogen vaccines), and may prevent side-effects associated with live attenuated vaccines. For instance, the World Health Organization recommends waiting nine months after birth before immunizing against rubella, measles, and mumps, in order to avoid undesirable side effects associated with vaccination against these diseases. Similarly, the World Health Organization recommends waiting two months after birth before immunizing children against influenza virus. In addition to concern over side effects, there is doubt as to whether an effective immune response may be generated using these conventional vaccines prior to the recommended ages.

In preferred embodiments of the invention, nucleic acids encoding more than one relevant epitope of one or more target antigen are administered to an infant mammal for the purposes of genetic immunization. It has been observed that the administration of several epitopes representing distinct target antigens of a pathogen provide a synergistic immune response to the pathogen. Similarly, the administration of multiple epitopes directed to antigens associated with more than one pathogen may be used to provide an infant subject with a broader spectrum of protection. Such an approach may be used to optimize the immunity induced, and may be a means for inducing an immune response to a variety of childhood pathogens.

### DESCRIPTION OF THE FIGURES

FIGS. 1A-D. Primary and secondary NP-specific cytotoxicity one month after injection of newborn (C-D) or adult (A-B) mice with DNA encoding influenza nucleoprotein (NPV1). The percentage of specific lysis was determined in a standard 4-hour ⁵¹Cr release assay for CTL (cytotoxic T lymphocytes) obtained from newborn or adult animals immunized with NPV1 or control DNA and boosted (or not) with live PR8 virus one month after completing the immunization. An additional control group was injected with saline and boosted one month later with virus. Spleen cells were harvested 7 days after boosting and the percentage of NP-specific cytotoxicity was determined immediately (i.e., primary cytotoxicity) or after incubation for five days with irradiated spleen cells, NP peptide, and IL-2 (i. e., secondary cytotoxicity) as described in Zaghouani et al., 1992, J. Immunol. 148:3604-3609. CTLs were assayed against P815 cells coated with NP peptide (5 μg/ml) or infected with PR8 (not shown) or B Lee virus.

FIGS. 2A-B. Limiting dilution assay to determine the frequency of NP-specific CTL precursors one month after injection of newborn (B) and adult (A) mice with NPV1. Splenocytes harvested 7 days after PR8 boosting from newborn and adult mice vaccinated with NPV1 or control plasmid were incubated in serial dilution (6×10⁴ to 2×10¹ splenocytes/well) for 5 days with x-irradiated, PR8-infected splenocytes from non-immunized BALB/c mice in the presence of IL-2 (6 units/ml). The incubation was carried out in 96-well microtiter plates with 24 wells for each dilution of effector cells. Cytotoxicity was assessed against PR8-infected or non-infected P815 cells. Those wells exhibiting percentage lysis greater than background plus three standard deviations were regarded as positive.

FIG. 3. Detection of DNA in muscle of BALB/c mice infected with NPV1. Muscle tissue was removed from the site of injection in the right gluteal muscle of newborns or tibial muscle of adults one month after completion of the vaccination schedule. DNA recovered from the muscle tissue on the left flank of each animal served as a control. The labeling above each lane indicates the origin of DNA. Lanes 1-4 represent adult right anterior tibial muscle; lane 5 represents adult left anterior tibial muscle; lanes 6-10 represent newborn right gluteal muscle; lane 11 represents newborn left gluteal muscle; lane 12 represents NPV1 plasmid; and lane 13 contains a DNA ladder.

FIGS. 4A-C. Cross-reactive CTLS generated in newborns injected with NPV1. The percentage of specific lysis was determined using a standard ⁵¹Cr release assay. Spleen cells were harvested from (A) PR8 immunized mice; (B) genetically immunized newborns that were immunized one month later with PR8 virus and (C) genetically immunized newborns Spleen cells were cultured for 4 days with irradiated PR8-infected spleen cells, then assayed in the presence of ⁵¹Cr-labeled P815 cells noninfected or infected with PR8, A/HK, A/Japan or B lee virus.

FIGS. 5A-F. Survival of genetically immunized newborn (C, D, E) and adult (A, B, F) mice challenged 1 mo. (A-D) or 3 mo. (E and F) after immunization with 1.5×10⁴ TCID₅₀ PR8 virus (A, C, E, F) or 3×10⁵ TCID₅₀ HK virus (B, D) via aerosol.

FIGS. 6A-B. Kinetics of body-weight loss and recovery in immunized adult (A) or newborn (B) mice challenged with 1.5×10⁴ TCID50 PR8 virus one month after completing the immunization.

FIG. 7A-D. Survival of (A-B) newborn and (C-D) adult mice immunized with pHA plasmid encoding hemagglutinin of WSN influenza virus and challenged with LD₁₀₀ of WSN (3×10⁷ TCID₅₀; A, C) or PR8 (1.5×10⁴ TCID₅₀; B, D) virus, 1 month after immunization.

FIGS. 8A-B. Cytotoxicity of splenocytes from mice immunized as neonates with either (A) UV-inactivated influenza virus or (B) pHA+pNP.

FIGS. 9A-C. Secretion of cytokines by CD4+ T cells from mice immunized with (A) pHA+pNP; (B) La-attenuated WSN virus; and (C) control.

FIGS. 10A-D. Survival of (A) newborn mice immunized with pHA+pNP, pHA, or pNP, challenged with WSN virus; (B) newborn mice immunized with pHA+pNP, pHA or pNP, challenged with PR8 virus; (C) adult mice immunized with pHA+pNP, pHA or pNP, challenged with WSN virus; (D) adult mice immunized with pHA+pNP, pHA or pNP following lethal challenge with PR8 virus.

FIG. 11. Relationship between number of inoculations and protection conferred.

FIG. 12. Lethality of various doses of WSN live virus in neonatal BALB/c mice.

FIG. 13. Survival of neonatal mice immunized with UV-attenuated WSN virus.

FIGS. 14A-B. Proliferation of CD4+ T cells (A) stimulated with NP 147-155 peptide or (B) from neonatal mice immunized with VH-TB, boosted with PR8 virus.

FIGS. 15A-D. Secretion of cytokines by T cells from (A) mice having received an inoculation with live PR8 virus, previously immunized (as adults) with VH-TB; (B) mice having received an inoculation with live PR8 virus, previously immunized (as neonates) with VH-TB; (C) mice having received an inoculation with live PR8 virus (no previous immunization); (D) mice having received an inoculation with live PR8 virus (no previous immunization as neonates).

FIG. 16. Cytotoxicity response of mice immunized as neonates with VH-TB. Mice (A) injected only with PR8 virus and (B) immunized with VH-TB and boosted with PR8 virus.

FIG. 17. Reactivity of polyclonal anti-IgG used in ELISA assays. Binding was assessed by direct ELISA against monkey IgG (mIgG) and human IgG, IgM or IgA (hIgG, hIgM, hIgA). The results expressed as OD405 nm versus concentration of immunoglobulin fractions used for coating, are representative for two independent experiments.

FIG. 18. Magnitude and kinetics of antibody titers in sera of baboons vaccinated as neonates with plasmids expressing influenza virus antigens. (A) WSN-specific IgG antibodies were measured by indirect ELISA and endpoint titers were estimated as corresponding to the highest dilution associated with a signal equal or above 3×background. Results are shown as means±SE of 1092 endpoint titers in various groups (hd—high dose; and—medium dose and Id—low dose). (B) WSN-specific HI antibodies were measured by standard inhibition of hemagglutination. The results are represented as means±SE of logz endpoint titers, corresponding to each group. Elisa titers less than 80 (A) and HI titers less than 40 (B) were neglected.

FIG. 19. Virus specific IgG antibodies in the sera of baboons inoculated as neonates with DNA vaccine (A, C) or control plasmid (B, D), before (A, B) or two weeks after (C, D) the instillation of live WSN virus at the age of one year and a half The results of the ELISA assay were expressed as absorption (mean OD405 nM, of duplicates) at various serum dilutions, representing binding to purified WSN virus (closed symbols) or background BSA (open symbols). The sera were simultaneously tested.

FIG. 20. Virus specific IgG antibodies in the nasal wash of baboons inoculated as neonates with DNA vaccine (A-E) or control plasmid (F-J) and subsequently exposed to live WSN virus via intratracheal inoculation. The results of the ELISA assay were expressed as absorption (mean OD405 nM of duplicates) at various serum dilutions, representing binding to purified WSN virus (closed symbols) or background BSA (open symbols). The samples harvested at various intervals after virus instillation, were simultaneously tested.

FIG. 21. In vitro neutralization of homologous virus by sera from baboons immunized as newborns with plasmid vaccine (group C) or controls (group D). (A) Inhibition of virus multiplication in permissive MDCK cells by sera harvested before (open bars) or 14 days after (closed bars) virus challenge. The results are expressed as endpoint titers corresponding to complete abrogation of virus multiplication. (B) Inhibition of antigen processing/presentation to specific TcH by sera harvested before (open bars) or 14 days after (closed bars) virus challenge. The results were expressed as endpoint titers corresponding to the highest dilution that inhibited the antigen presentation.

### DETAILED DESCRIPTION OF THE INVENTION

For purposes of clarity of description, and not by way of limitation, the detailed description of the invention is divided into the following subsections:

(i) compositions for immunization; and

(ii) methods of immunization.

### Compositions for Immunization

The present invention provides for compositions which may be used to immunize infant mammals against one or more target antigens which comprise an effective amount of a nucleic acid encoding one or more relevant epitopes of the target antigen(s) in a pharmaceutically acceptable carrier. Following administration of the compositions, transformed host cells will express the relevant antigens, thereby provoking the desired immune response.

Nucleic acids which may be used herein include deoxyribonucleic acid (“DNA”) as well as ribonucleic acid (“RNA”). It is preferable to use DNA in view of its greater stability to degradation.

The term “target antigen” refers to an antigen toward which it is desirable to induce an immune response. Such an antigen may be comprised in a pathogen, such as a viral, bacterial, protozoan, fungal, yeast, or parasitic antigen, or may be comprised in a cell, such as a cancer cell or a cell of the immune system which mediates an autoimmune disorder. For example, but not by way of limitation, the target antigen may be comprised in an influenza virus, a cytomegalovirus, a herpes virus (including HSV-I and HSV-II), a vaccinia virus, a hepatitis virus (including but not limited to hepatitis A, B, C, or D), a varicella virus, a rotavirus, a papilloma virus, a measles virus, an Epstein Barr virus, a coxsackie virus, a polio virus, an enterovirus, an adenovirus, a retrovirus (including, but not limited to, HIV-1 or HIV-2), a respiratory syncytial virus, a rubella virus, a Streptococcus bacterium (such as Streptococcus pneumoniae), a Staphylococcus bacterium (such as Staphylococcus aureus), a Hemophilus bacterium (such as Hemophilus influenzae), a Listeria bacterium (such as Listeria monocytogenes), a Klebsiella bacterium, a Gram-negative bacillus bacterium, an Escherichia bacterium (such as Escherichia coli), a Salmonella bacterium (such as Salmonella typhimurium), a Vibrio bacterium (such as Vibrio cholerae),a Yersinia bacterium (such as Yersinia pestis or Yersinia enterocoliticus), an Enterococcus bacterium, a Neisseria bacterium (such as Neiserria meningitidis), a Corynebacterium bacterium (such as Corynebacterium diphtheriae), a Clostridium bacterium (such as Clostridium tetani), a Mycoplasma (such as Mycoplasma pneumoniae), a Pseudomonas bacterium, (such as Pseudomonas aeruginosa), a Mycobacteria bacterium (such as Mycobacterium tuberculosis), a Candida yeast, an Aspergillus fungus, a Mucor fungus, a toxoplasma, an amoeba, a malarial parasite, a trypanosomal parasite, a leishmanial parasite, a helminth, etc. Specific nonlimiting examples of such target antigens include hemagglutinin, nucleoprotein, M protein, F protein, HBS protein, gp120 protein of HIV, nef protein of HIV, and listeriolysine. In alternative embodiments, the target antigen may be a tumor antigen, including, but not limited to, carcinoembryonic antigen (“CEA”), melanoma associated antigens, alpha fetoprotein, papilloma virus antigens, Epstein Barr antigens, etc..

The term “relevant epitope”, as used herein, refers to an epitope comprised in the target antigen which is accessible to the immune system. For example, a relevant epitope may be processed after penetration of a microbe into a cell or recognized by antibodies on the surface of the microbe or microbial proteins. Preferably, an immune response directed toward the epitope confers a beneficial effect; for example, where the target antigen is a viral protein, an immune response toward a relevant epitope of the target antigen at least partially neutralizes the infectivity or pathogenicity of the virus. Epitopes may be B-cell or T-cell epitopes.

The term “B cell epitope”, as used herein, refers to a peptide, including a peptide comprised in a larger protein, which is able to bind to an immunoglobulin receptor of a B cell and participates in the induction of antibody production by the B cells.

For example, and not by way of limitation, the hypervariable region 3 loop (“V3 loop”) of the envelope protein of human immunodeficiency virus (“HIV”) type 1 is known to be a B cell epitope. Although the sequence of this epitope varies, the following consensus sequence, corresponding to residues 301-319 of HIV-1 gp120 protein, has been obtained: Arg-Lys-Ser-Ile-His-Ile-Gly-Pro-Gly-Arg-Ala-Phe-Tyr-Thr-Thr-Gly-Glu-Ile-Ile (SEQ ID NO:1).

Other examples of known B cell epitopes which may be used according to the invention include, but are not limited to, epitopes associated with influenza virus strains, such as Trp-Leu-Thr-Lys-Lys-Gly-Asp-Ser-Tyr-Pro (SEQ ID NO:2), which has been shown to be an immunodominant B cell epitope in site B of influenza HA1 hemagglutinin, the epitope Trp-Leu-Thr-Lys-Ser-Gly-Ser-Thr-Tyr-Pro (H3; SEQ ID NO:3), and the epitope Trp-Leu-Thr-Lys-Glu-Gly-Ser-Asp-Tyr-Pro (H2; SEQ ID NO:4) (Li et al., 1992, J. Virol. 66:399-404); an epitope of F protein of measles virus (residues 404-414; Ile-Asn-Gln-Asp-Pro-Asp-Lys-Ile-Leu-Thr-Tyr; SEQ ID NO:5; Parlidos et al., 1992, Eur. J. Immunol. 22:2675-2680); an epitope of hepatitis virus pre-SI region, from residues 132-145 (Leclerc, 1991, J. Immunol. 147:3545-3552); and an epitope of foot and mouth disease VP1 protein, residues 141-160, Met-Asn-Ser-Ala-Pro-Asn-Leu-Arg-Gly-Asp-Leu-Gln-Lys-Val-Ala-Arg-Thr-Leu-Pro (SEQ ID NO:6; Clarke et al., 1987, Nature 330:381-384).

Still further B cell epitopes which may be used are known or may be identified by methods known in the art, as set forth in Caton et al., 1982, Cell 31:417-427.

In additional embodiments of the invention, peptides which may be used according to the invention may be T cell epitopes. The term “T cell epitope”, as used herein, refers to a peptide, including a peptide comprised in a larger protein, which may be associated with MHC self antigens and recognized by a T cell, thereby functionally activating the T cell.

For example, the present invention provides for Tₕ epitopes, which, in the context of MHC class II self antigens, may be recognized by a helper T cell and thereby promote the facilitation of B cell antibody production via the Tₕ cell.

For example, and not by way of limitation, influenza A hemagglutinin (HA) protein of PR8 strain, bears, at amino acid residues 110-120, a Tₕ epitope having the amino acid sequence Ser-Phe-Glu-Arg-Phe-Glu-Ile-Phe-Pro-Lys-Glu (SEQ ID NO:7).

Other examples of known T cell epitopes include, but are not limited to, two promiscuous epitopes of tetanus toxoid, Asn-Ser-Val-Asp-Asp-Ala-Leu-Ile-Asn-Ser-Thr-Lys-Ile-Tyr-Ser-Tyr-Phe-Pro-Ser-Val (SEQ ID NO:8) and Pro-Glu-Ile-Asn-Gly-Lys-Ala-Ile-His-Leu-Val-Asn-Asn-Glu-Ser-Ser-Glu (SEQ ID NO:9; Ho et al., 1990, Eur. J. Immunol. 20:477-483); an epitope of cytochrome c, from residues 88-103, Ala-Asn-Glu-Arg-Ala-Asp-Leu-Ile-Ala-Tyr-Leu-Gln-Ala-Thr-Lys (SEQ ID NO:10); an epitope of Mycobacteria heatshock protein, residues 350-369, Asp-Gln-Val-His-Phe-Gln-Pro-Leu-Pro-Pro-Ala-Val-Val-Lys-Leu-Ser-Asp-Ala-Leu-Ile (SEQ ID NO:11; Vordermir et al., Eur. J. Immunol. 24:2061-2067); an epitope of hen egg white lysozyme, residues 48-61, Asp-Gly-Ser-Thr-Asp-Tyr-Gly-Ile-Leu-Gln-Ile-Asn-Ser-Arg (SEQ ID NO:12; Neilson et al., 1992, Proc. Natl. Acad. Sci. U.S.A. 89:7380-7383); an epitope of Streptococcus A M protein, residues 308-319, Gln-Val-Glu-Lys-Ala-Leu-Glu-Glu-Ala-Asn-Ser-Lys (SEQ ID NO:13; Rossiter et al., 1994, Eur. J. Immunol. 24:1244-1247); and an epitope of Staphylococcus nuclease protein, residues 81-100, Arg-Thr-Asp-Lys-Tyr-Gly-Arg-Gly-Leu-Ala-Tyr-Ile-Tyr-Ala-Asp-Gly-Lys-Met-Val-Asn (SEQ ID NO: 14; de Magistris, 1992, Cell 68:1-20). Still further Th epitopes which may be used are known or may be identified by methods known in the art.

As a further example, a relevant epitope may be a T_{CTL} epitope, which, in the context of MHC class I self antigens, may be recognized by a cytotoxic T cell and thereby promote CTL-mediated lysis of cells comprising the target antigen. Nonlimiting examples of such epitopes include epitopes of influenza virus nucleoproteins TYQRTRALVRTGMDP (SEQ ID NO:15) or IASNENMDAMESSTL (SEQ ID NO:16) corresponding to amino acid residues 147-161 and 365-379, respectively (Taylor et al., 1989 Immunogenetics 26:267; Townsend et al., 1983, Nature 348:674); LSMV peptide, KAVYNFATM, amino acid residues 33-41 (SEQ ID NO:17; Zinhernagel et al., 1974, Nature 248:701-702); and oval bumin peptide, SIINFEKL, corresponding to amino acid residues 257-264 (SEQ ID NO:18; Cerbone et al., 1983, J. Exp. Med. 163:603-612).

The nucleic acids of the invention encode one or more relevant epitopes, and may optionally further comprise elements that regulate the expression and/or stability and/or immunogenicity of the relevant epitope. For example, elements that regulate the expression of the epitope include, but are not limited to, a promoter/enhancer element, a transcriptional initiation site, a polyadenylation site, a transcriptional termination site, a ribosome binding site, a translational start codon, a translational stop codon, a signal peptide, etc. Specific examples include, but are not limited to, a promoter and intron A sequence of the initial early gene of cytomegalovirus (CMV or SV40 virus (“SV40”); Montgomery et al., 1993, DNA and Cell Biology 12:777-783). With regard to enhanced stability and/or immunogenicity of the relevant epitope, it may be desirable to comprise the epitope in a larger peptide or protein. For example, and not by way of limitation, the relevant epitope may be comprised in an immunoglobulin molecule, for example, as set forth in U.S. patent application Ser. No. 08/363,276, by Bona et al., the contents of which is hereby incorporated in its entirety herein by reference. Alternatively, more than one epitope may be expressed within the same open reading frame.

Nucleic acids encoding the relevant epitope(s) and optionally comprising elements that aid in its expression, stability, and/or immunogenicity may be comprised in a cloning vector such as a plasmid, which may be propagated using standard techniques to produce sufficient quantities of nucleic acid for immunization. The entire vector, which may preferably be a plasmid which is a mammalian expression vector comprising the cloned sequences, may be used to immunize the infant animal. Sequences encoding more than one epitope of one or more target antigens may be comprised in a single vector.

Examples of nucleic acids which may be used according to the invention are set forth in International Application Publication No. WO 94/21797, by Merck & Co. and Vical, Inc., U.S. Pat. Nos. 5,589,466 and 5,580,859 and in International Application Publication No. WO 90/11092, by Vical, Inc., the contents of which are hereby incorporated in their entireties herein by reference.

Different species of nucleic acid, encoding more than one epitope of one or more target antigens, may be comprised in the same composition or may be concurrently administered as separate compositions. The term “different species”, as used herein, refers to nucleic acids having different primary sequences. For example, a composition of the invention may comprise one species of nucleic acid encoding a first epitope and a second species of nucleic acid encoding a second epitope, with mulitple molecules of both species being present.

The term “effective amount”, as used herein, refers to an amount of nucleic acid encoding at least one relevant epitope of at least one target antigen, which, when introduced into a infant mammal, results in a substantial increase in the immune response of the mammal to the target antigen. Preferably, the cellular and/or humoral immune response to the target antigen is increased, following the application of methods of the invention, by at least four-fold, and preferably by at least between 10-fold and 100-fold (inclusive), above baseline. The immunity elicited by such genetic immunization may develop rapidly after the completion of the immunization (e.g., within 7 days), and may be long lasting (e.g., greater than 9 months). The need for “boosting” in order to achieve an effective immune response may be diminished by the present invention. In preferred embodiments, the effective amount of nucleic acid is introduced by multiple inoculations (see below).

In specific, nonlimiting embodiments of the invention, nucleic acid encoding between 1-500 picomoles of relevant epitope, preferably between 20-100 picomoles of relevant epitope, and more preferably between 40-100 picomoles of relevant epitope per gram weight of the infant mammal may be administered.

As demonstrated in the working examples set forth below, DNA immunization of new born baboons resulted in induction of immune memory. The baboons were inoculated with either 40 μg/plasmid, 200 μg/plasmid or 1 mg/plasmid per dose.

The baboons inoculated with the highest dose of plasmid showed the highest titres of protective antibodies. Thus in a preferred embodiment of the invention, infants will be inoculated with doses of plasmid equivalent to those used for immunization of baboons. The amount of plasmid to be inoculated into an infant can be calculated by determining the ratio between the average weight of a baboon and that of an infant and applying that ratio to the amount of inoculating DNA. Those of skill in the art can readily extrapolate the doses required for inoculation of infants from those amounts used in baboons.

Thus, in selected embodiments the compositions of the present invention may comprise strands of nucleic acids encoding more than one relevant epitope. As explained herein, the relevant epitopes may be found in the same target antigen, in different antigens from the same pathogen or in unrelated target antigens from different pathogens. With respect to the latter, opportunistic pathogens may be targeted along with the primary disease causing agent. In addition to the broad target range, the disclosed compositions may comprise various epitope combinations. For example, the compositions of the present invention may comprise nucleic acids encoding mixtures of B cell epitopes, mixtures of T cell epitopes, or combinations of B and T cell eptiopes. Regardless of which type of epitopes are selected, it will be appreciated that the relevant epitopes may be encoded on the same nucleic acid molecule (i.e., a plasmid) and may even be expressed within the same open reading frame. Alternatively, relevant epitopes may be encoded by separate, non-covalently bound nucleic acid molecules which may be administered in combination as a vaccine “cocktail”. In particularly preferred embodiments these combination vaccines will comprise one or more species of plasmid, each encoding at least one relevant epitope.

As will be demonstrated by the appendant examples, genetic vaccination of infants using compositions comprising nucleic acid molecules (whether as a single species or as a combination of species) which express more than one relevant epitope may exhibit an unexpected synergistic effect. More particularly, such combination vaccines may prove to be much more efficient at conferring the desired immunity with respect to the selected pathogen(s) than compositions comprising a single nucleic acid species encoding a single relevant epitope. Those skilled in the art will appreciate that such synergism could allow for an effective immunoprophylactic or immunotherapeutic response to be generated with lower dosing and less frequent administration than single-epitope DNA vaccines. Moreover, the use of such multi-epitope DNA vaccine compositions may provide more comprehensive protection as the induced multi-site immunity would tend to be more resistant to natural phenotypic variation within a species or rapid mutation of a target antigen by the selected pathogen. Of course, effective immunity may also be imparted by DNA vaccines encoding a single B or T cell epitope and such compositions are clearly contemplated as being within the scope of the present invention.

In addition to nucleic acids, the compositions of the invention may comprise a pharmaceutically acceptable carrier, such as, for example, but not limited to, physiologic saline or liposomes. In specific, nonlimiting embodiments, the concentration of nucleic acid preferably ranges from 30-100 μg/100 μl. In certain embodiments, it may be desirable to formulate such compositions as suspensions or as liposomal formulations.

### Methods of Immunization

The present invention provides for a method for immunizing an infant mammal against one or more target antigen, comprising inoculating the mammal with an effective amount of nucleic acid(s) encoding relevant epitope(s) of the target antigen(s) in a pharmaceutically acceptable carrier.

The term “infant”, as used herein, refers to a human or non-human mammal during the period of life following birth wherein the immune system has not yet fully matured. In humans, this period extends from birth to the age of about nine months, inclusive. In mice, this period extends from birth to about four weeks of age. The terms “newborn” and “neonate” refer to a subset of infant mammals, which have essentially just been born. Other characteristics associated with “infants” according to the invention include an immune response which has (i) susceptibility to high zone tolerance (deletion/anergy of T cell precursors, increased tendency for apoptosis); (ii) a Th2 biased helper response (phenotypical particularities of neonatal T cells; decreased CD40L expression on neonatal T cells); (iii) reduced magnitude of the cellular response (reduced number of functional T cells; reduced antigen-presenting cell function); and (iv) reduced magnitude and restricted isotype of humoral response (predominance of IgM^{high} IgD^{low} B cells, reduced cooperation between Th and B cells). In specific nonlimiting embodiments of the invention, nucleic acid immunization may be administered to an infant animal wherein maternal antibodies remain present in detectable amounts.

In specific nonlimiting embodiments of the invention, nucleic acid immunization may be administered to an infant mammal wherein maternal antibodies remain present in detectable amounts. In a related embodiment, the pregnant mother may be immunized with a nucleic-acid based vaccine prior to delivery so as to increase the level of maternal antibodies passively transferred to the fetus.

The terms “immunize” or “immunization” or related terms refer herein to conferring the ability to mount a substantial immune response (consisting of antibodies or cellular immunity such as effector CTL) against a target antigen or epitope. These terms do not require that completely protective immunity be created, but rather that a protective immune response be produced which is substantially greater than baseline. For example, a mammal may be considered to be immunized against a target antigen if the cellular and/or humoral immune response to the target antigen occurs following the application of methods of the invention. Preferably, immunization results in significant resistance to the disease caused or triggered by pathogens expressing target antigens.

The term “inoculating”, as used herein, refers to introducing a composition comprising at least one species of nucleic acid according to the invention into a infant animal. As mentioned above, the composition may comprise more than one nucleic acid species directed to one or more relevant epitopes found on one or more target antigen. The introduction of the selected composition may be accomplished by any means and route known in the art, including intramuscular, subcutaneous, intravenous, intraperitoneal, intrathecal, oral, nasal, rectal, etc. administration. Preferably, inoculation is performed by intramuscular injection.

The effective amount of nucleic acid is preferably administered in several inoculations (that is to say, the effective amount may be split into several doses for inoculation). The number of inoculations is preferably at least one, and is more preferably three.

The success of the inoculations may be confirmed by collecting a peripheral blood sample from the subject between one and four weeks after immunization and testing for the presence of CTL activity and/or a humoral response directed against the target antigen, using standard immunologic techniques.

In specific, nonlimiting embodiments, the present invention may be used to immunize a human infant as follows. A human infant, at an age ranging from birth to about 9 months, preferably at an age ranging from birth to about 6 months, more preferably at an age ranging from birth to about 1 month, and most preferably at an age ranging from birth to about 1 week, may commence a program of injections whereby the infant may be injected intramuscularly three times at 3-7 day intervals with a composition comprising 1-100 nanomoles of DNA encoding a relevant epitope(s) of target antigen(s), preferably at a DNA concentration of 1-5 mg/100 μl, wherein the target antigen may be a protein from a pathogen, for example respiratory syncytial virus, rotavirus, influenza virus, hepatitis virus, or HIV virus (see above).

In addition, a two step immunization protocol may be used wherein neonatal priming with DNA vaccine is followed by subsequent boosts with conventional vaccines, such as live virus at an older age.

Accordingly, the present invention provides for compositions for use in immunizing an infant mammal against one or more target antigens, comprising one or more species of nucleic acid encoding one or more epitopes of said target antigen(s) in an amount effective in inducing a cellular (e.g. CTL) and/or humoral immune response.

It is believed that one of the advantages of the present invention is that mammals immunized by such methods may exhibit a lesser tendency to develop an allergy or other adverse reaction after exposure to target antigens. Further, DNA vaccination of infants may reduce the risk of tolerance induction following other vaccination protocols which require successive administration of relatively high doses of antigen.

In preferred embodiments (see Example 7, infra), the present invention provides for a method for immunizing an infant animal against one or more pathogen comprising inoculating the mammal with an effective amount of nucleic acid(s) encoding more than one relevant epitope of one or more target antigen associated with the pathogen(s) in a pharmaceutically acceptable carrier, such that therapeutically effective amounts of the relevant epitopes are expressed in the infant mammal. Analogous methods may be used to induce immunity to undesirable cells or organisms which are not pathogens.

### EXAMPLE

### Induction of Cellular Immunity Against Influenza Virus Nucleoprotein in Newborn Mice by Genetic Vaccination

### Materials and Methods

Plasmids. The NPV1 plasmid (obtained from Dr. Peter Palese) was constructed by inserting a cDNA derived from the nucleoprotein gene of A/PR8/34 into the BglII site of a mutated pBR322 vector, namely pCMV-IE-AKi-DHFR (Whong et al., 1987, J. Virol. 61:1796), downstream from a 1.96 kb segment of the enhancer, promoter and intron A sequence of the initial early gene of cytomegalovirus and upstream of a 0.55 kb segment of the β globin polyadenylation signal sequence as described in Ulmer et al., 1993, Science 259:1745. The modified pBR322 vector without the NP sequence (termed the “V1 plasmid”) was employed as a control. PRc/CMV-HA WSN plasmid (pHA plasmid or WSN-HA plasmid) was constructed by inserting HA of A/WSN/33 (subtype H1N1) strain of influenza virus into the PRc/CMV mammalian expression vector and donated by Dr. Peter Palese (Mount Sinai School of Medicine). All plasmids were propagated in Escherichia coli and purified by the alkaline lysis method (Id.).

Viruses. The influenza virus strains A/PR8/34 (H1N1), A/HK/68(H3N2), A/Japan/305/57(H2N2) and B Lee/40 were grown in the allantoic cavity of embryonated hen eggs as described in Kilbourne, 1976, J. Infect. Dis. 134:384-394. The A/HK/68 virus adapted to mice was provided by Dr. Margaret Liu (Merck Research Laboratories). The influena virus strain A/WSN/33 was grown in MDBK cells and purified from supernatants.

Immunization. One month old adult mice were vaccinated with 30 μg of NPV1, pHA or control plasmid dissolved in 100 μl of physiologic saline by injection into the anterior tibial muscle of the shaved right leg using a disposable 28 gauge insulin syringe that was permitted to penetrate to a depth of 2 mm; three injections with 30 μg DNA were carried out at three week intervals. Newborn mice were immunized with 30 μg of plasmid dissolved in 50 μl of physiologic saline by similar injection into the right gluteal muscle of Days 1, 3 and 6 after birth of life. Some newborn mice were injected intraperitoneally (“IP”) on Day 1 after birth with PR8 or B Lee live virus (5 μg in 0.1 ml saline). One month after completion of the vaccination schedule, some mice were boosted with live virus in saline at a dose of 1×10³ TCID₅₀ injected ip.

Infection. Mice were challenged via the aerosol route with 1.5×10⁴ TCID₅₀ of A/PR8/34 (LD100) or 3.2×10⁵ TCID₅₀ of A/HK/68 (LD₁₀₀ virus) or 3×10⁷ TCID₅₀ of A/WSN/33 (LD₁₀₀). Exposure was carried out for 30 minutes in an aerosol chamber to which a nebulizer (Ace Glass, Inc.) was attached via a vacuum/pressure system pump operated at a rate of 35 L/min and a pressure of 15 lb/in². Mice were observed once daily post-infection and their survival was recorded.

Viral lung titers. Processing of lung tissue was carried out with at least three mice from each treatment group as described in as described in Isobe et al., 1994, Viral Immunol. 7:25-30, and viral titers in lung homogenates were determined using an MDCK cell-chicken RBC hemagglutination assay.

Cytotoxic assay. A primary cytotoxicity assay was carried out by incubating effector cells with 5×10^{3 51}Cr-labeled target cells at different effector-to-target ratios in 96-well V-bottom plates. P815 target cells were infected with PR8 virus for 1 hour before labeling with ⁵¹Cr or incubated during the assay with 5-10 μg/ml of NP₁₄₇₋₁₅₅. After incubation for 4 hours at 37° C. in 5% CO₂, the supernatant was harvested and radioactivity released was determined using a gamma counter. A secondary cytotoxicity assessment was carried out after co-culturing equal numbers of lymphocytes from test animals and x-irradiated, virus-infected or NP₁₄₇₋₁₅₅-coated lymphocytes from non-immunized BALB/c mice for five days in RPMI supplemented with fetal calf serum (“FCS”) 10% and 50 μM 2-mercaptoethanol; the secondary CTL assay itself was conducted using the ⁵¹Cr release assay described above, and the results were expressed as the percentage of specific lysis determined in triplicate for each effector:target ratio employed, as follows:

100(actual−spontaneous release)÷(maximum−spontaneous release−background release)±SD

Limiting dilution analysis of CTL precursors. The number of antigen-specific CTL precursors in the spleens of immunized mice were assessed by incubating single-cell suspensions of splenic responder cells in six steps of two-fold dilutions with 2.5×10⁵ X-irradiated, PR8-infected syngeneic splenocytes. After five days in complete RPMI medium, individual microtiter cultures were assayed using ⁵¹Cr release from P815 cells infected with influenza virus; uninfected P815 cells were used as a control. Those wells exhibiting ⁵¹Cr release greater than background plus three standard deviations were regarded as positive. The percentage of cultures in one dilution step regarded as negative for specific cytotoxicity were plotted logarithmically against the number of responder cells/well, and the frequency of CTL precursors was determined by linear regression analysis using the following formula:

−1n(negative-well index)÷(number of responder cells/well)=1/(number of responder cells/well at 0.37 negative well index).

The number of precursor cells is represented as 1/frequency for purposes of comparison.

Plasmid detection by PCR. Injected and control muscle tissue was removed one month after completion of the vaccination schedule, immediately frozen in ethanol-dry ice, and stored at −80° C. Frozen tissue was homogenized in lysis buffer and DNA was extracted as described in Montgomery, 1993, DNA and Cell Biol. 12:777-783 and Ulmer et al., 1993, Science 254:1745. A forty-cycle PCR reaction was carried out with NP-specific primers located at the following nucleotide positions: 1120 (minus strand; 5′-[CATTGTCTAGAATTTGAACTCCTCTAGTGG]-3′; SEQ ID NO:19) as well as 468 (positive strand; 5′-[AATTTGAATGATGCAAC]-3′; SEQ ID NO:20). A PCR product with a specific signal of 682 bp was visualized using ethidium bromide stained agarose gels.

Hemagglutination inhibition assay. Sera from immunized mice were treated with receptor destroying enzyme (RDE/neuraminidase) for 1 hour at 37° C. in a waterbath. Two-fold serial dilutions of RDE-treated sera were incubated with 0.5% human erythrocyte saline suspension in the presence of hemagglutinating titers of influenza virus. The experiment was carried out in triplicate wells. After 45 minutes incubation in a 96-well round bottom RIA plates (Falcon) at room temperature, the results were read and expressed as log₂ of the last inhibitory dilution. Negative controls (blank sera) and positive controls (HA specific monoclonal antibodies) were included in the experiment.

Cytokine measurement by ELISA. T cells were incubated, for four days, with antigen and irradiated accessory cells, and then 100 microliters of supernatant were harvested from each microculture. The concentrations of IFN gamma and IL-4 were measured using ELISA test kits (Cytoscreen, from Biosource Int. and Interest from Genzyme, respectively). Standards with known concentrations were included in the assay. The optical densities were assessed at 450 nm absorbance after blanking the ELISA read on the null concentration wells.

### Results

Priming of CTL precursors via neonatal DNA vaccination. The optimal schedule for DNA vaccination in the experiments described was developed in pilot studies. Newborn mice were immunized with 30 μg of NPV1 or control plasmid on Days 1, 3 and 6 after birth; adult animals were vaccinated with the same amount of DNA immunogen on Days 0, 21 and 42 of the study. One month after the completion of this standard series of vaccinations, certain test animals were boosted with live PR8 virus.

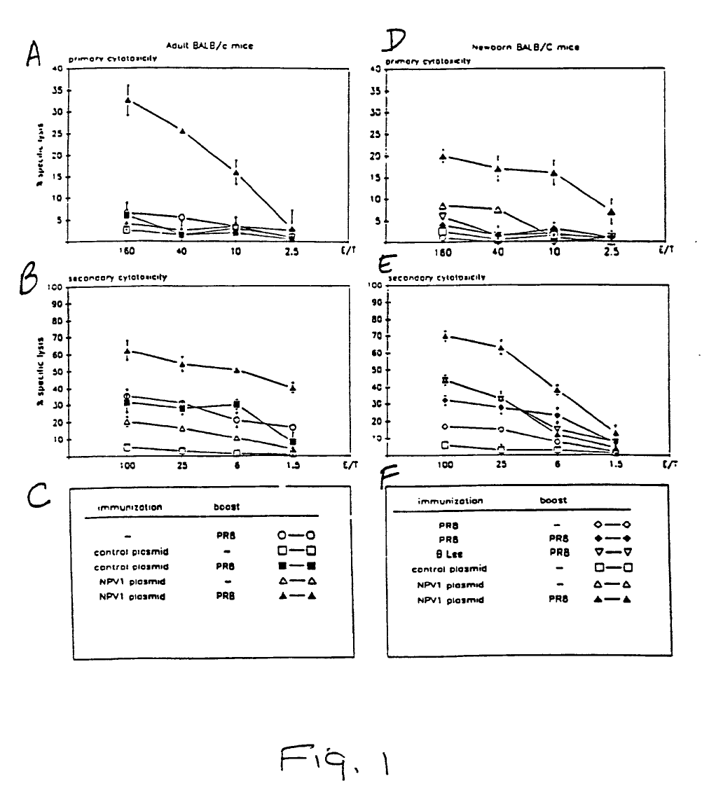

The lymphocytes, directly isolated from newborn and adult mice vaccinated with NPV1 and boosted with PR8 virus, lysed target cells coated in vitro with NP₁₄₇₋₁₅₅, which is recognized by CTL in association with K^{d} MHC-molecules of Class I (FIGS. 1A-D). No primary cytotoxicity was observed in vitro with lymphocytes from newborns immunized on Day 1 with PR8 virus and boosted one month later with PR8 virus. As expected, significant cytotoxicity was observed after in vitro expansion of splenocytes from mice immunized with NP-V1 plasmid or PR8 virus only. No significant cytotoxicity was observed in the case of mice immunized with control virus or B/Lee virus. These data clearly indicate that vaccination with NPV1 with or without subsequent boosting with native virus induced an expansion of NP-specific CTL precursors in both newborn animals and adults; however, both primary cytotoxicity and immunologically significant secondary cytotoxicity were observed only in animals fully immunized with NPV1 and boosted with virus.

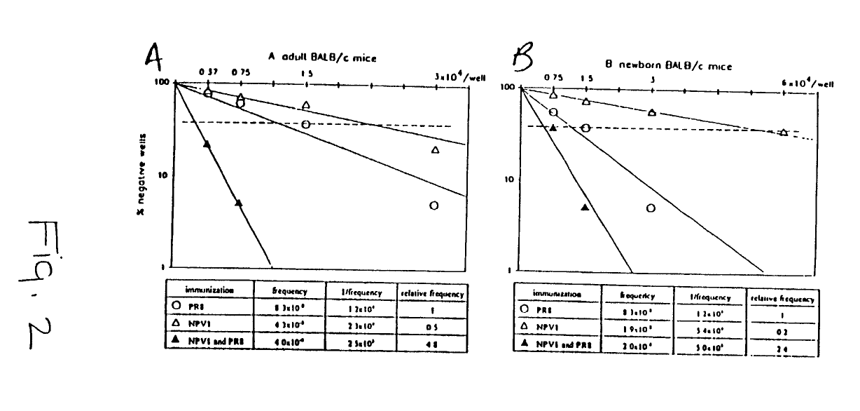

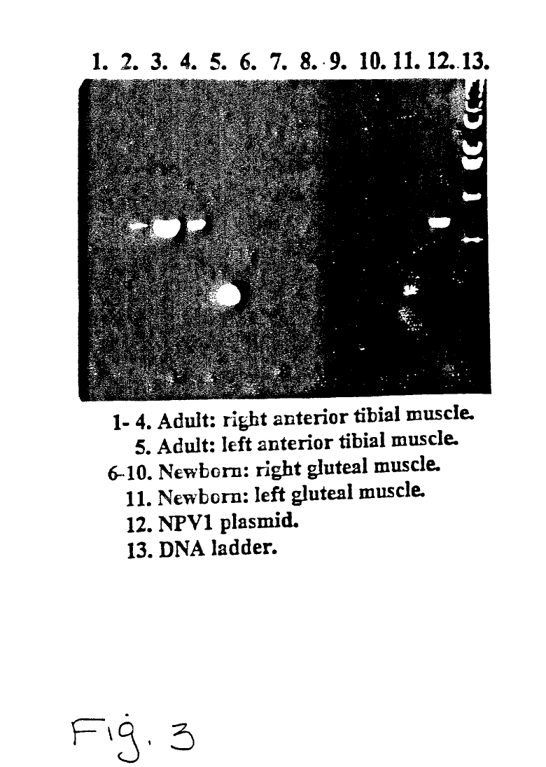

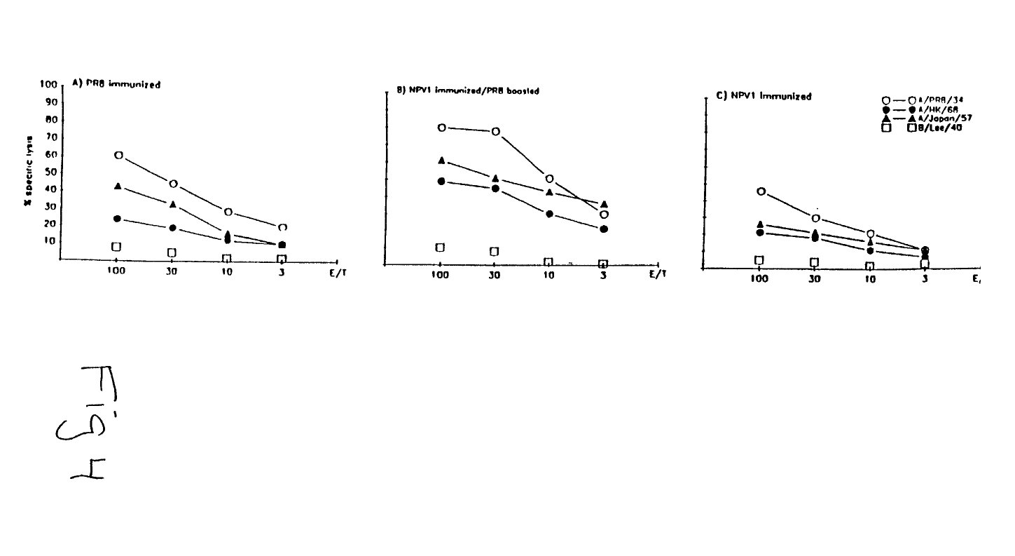

Frequency of NP-specific CTL precursors. An immunologically significant increase in the frequency of NP-specific CTL precursors was observed in animals immunized with NPV1 and boosted with PR8 virus, accounting for the presence of primary cytotoxicity in this particular group (FIGS. 2A-B). The increased frequency of specific precursors is presumably due to sustained biosynthesis of NP antigen, which primed and expanded this population of NP-specific lymphocytes. Plasmid was detected by qualitative PCR one month after completion of the immunization series in gluteal muscle, the site of injection of NPV1 in newborns, and in tibial muscle, the site of injection of NPV1 in adult animals (FIG. 3).

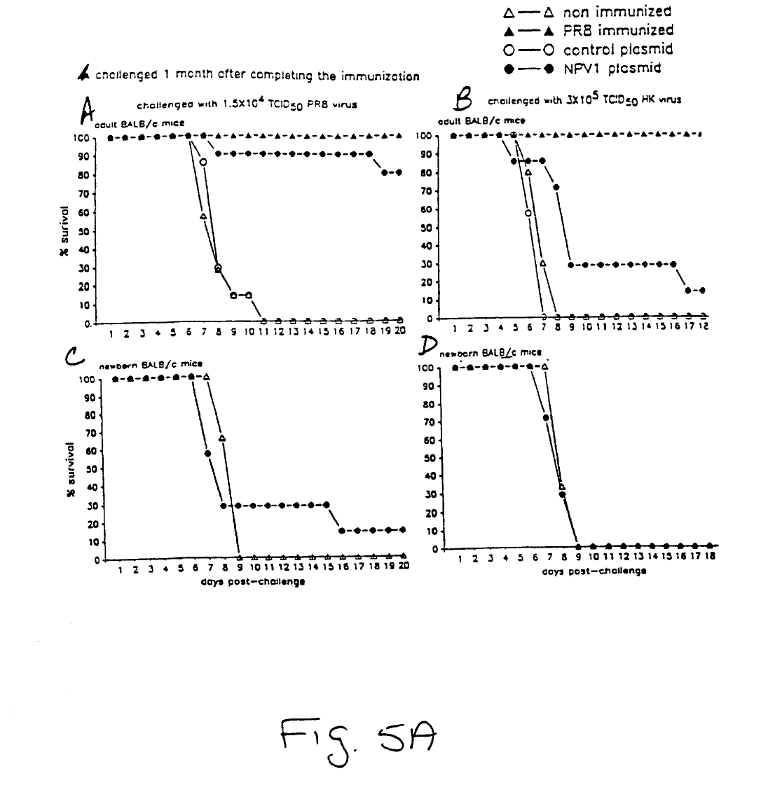

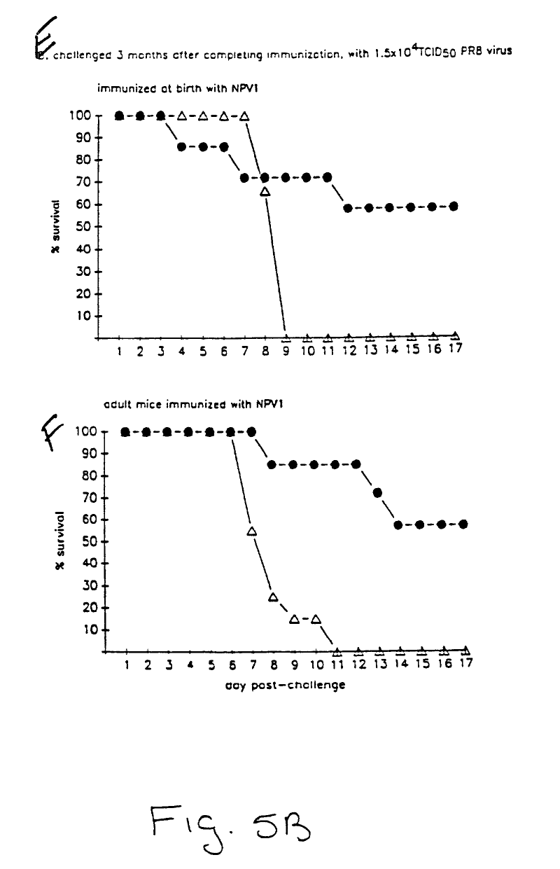

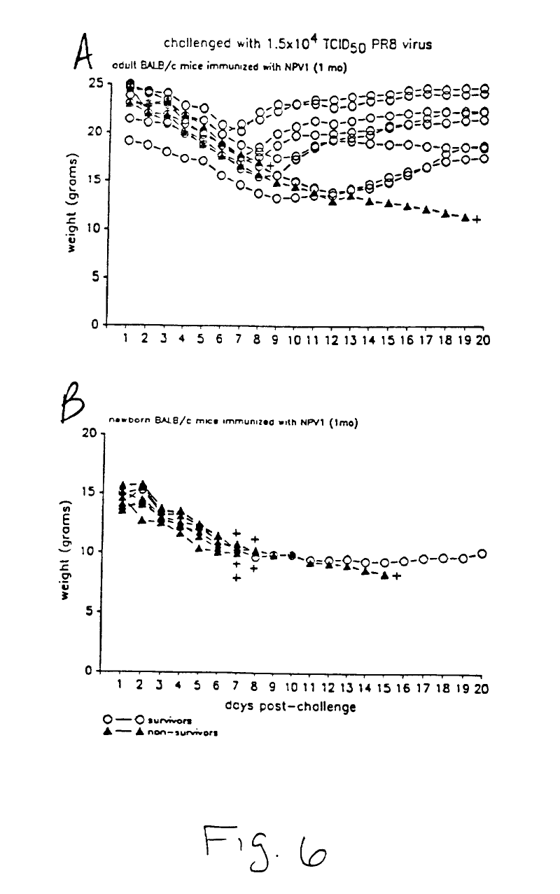

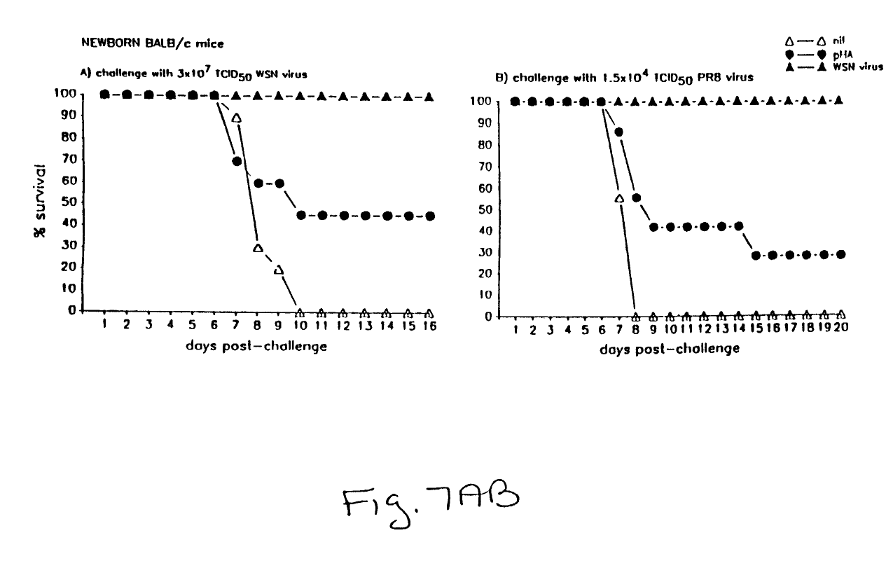

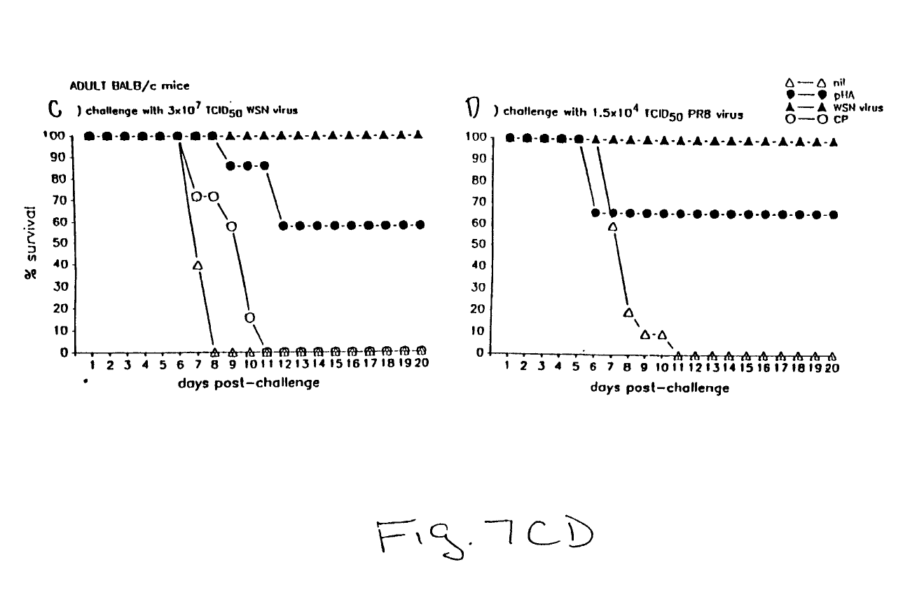

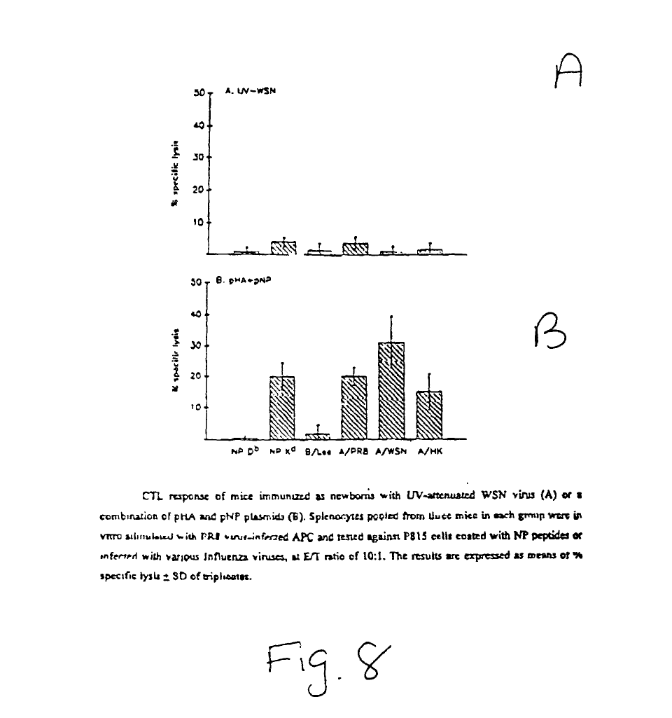

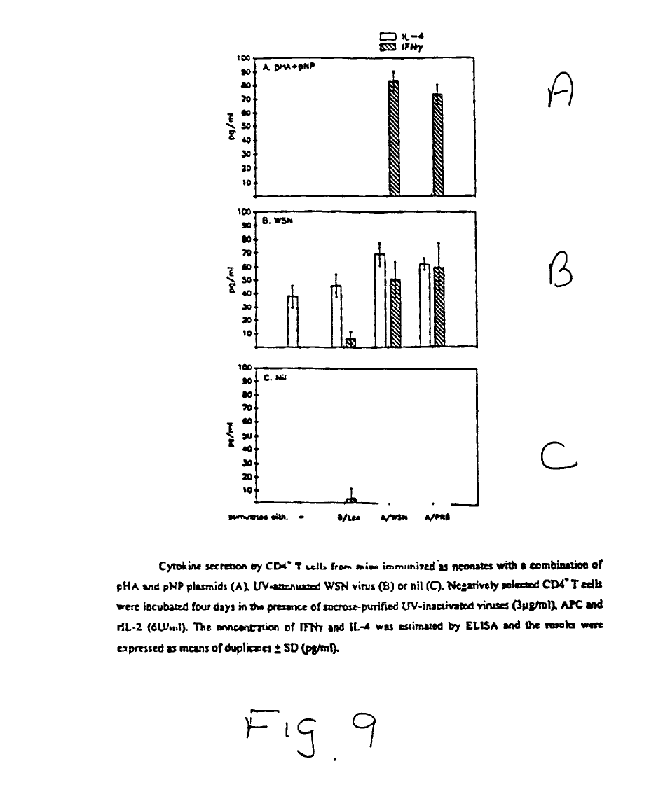

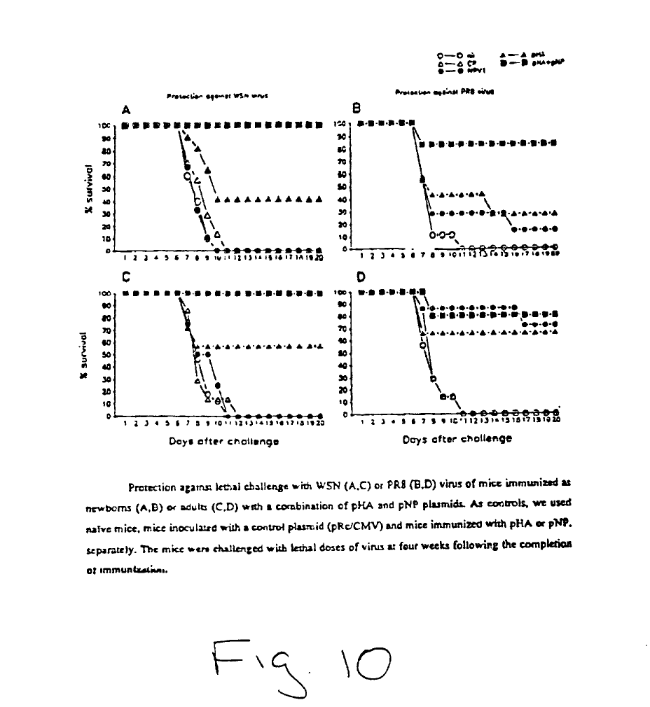

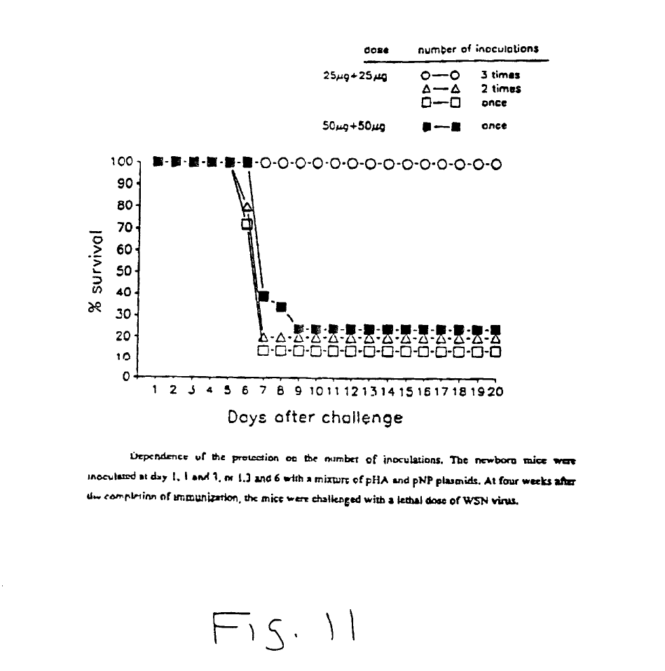

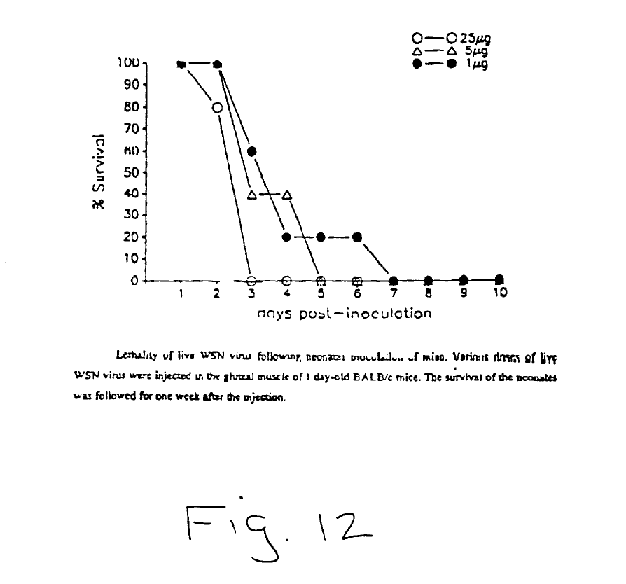

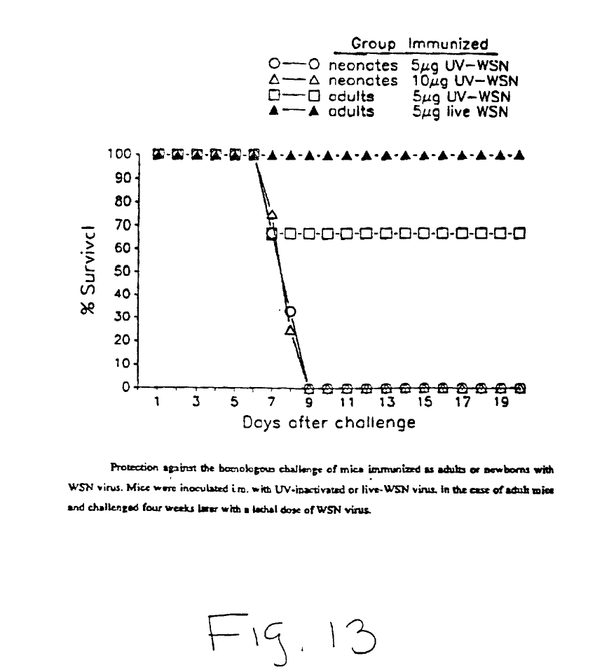

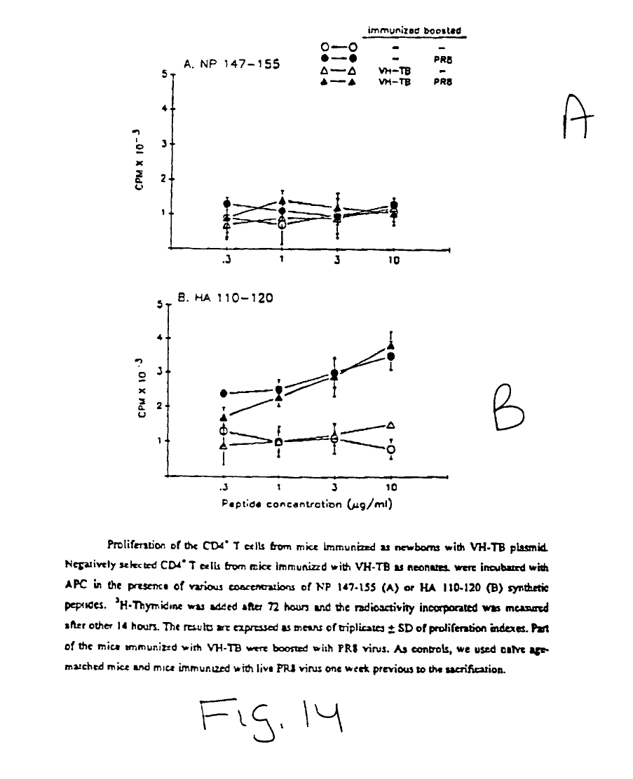

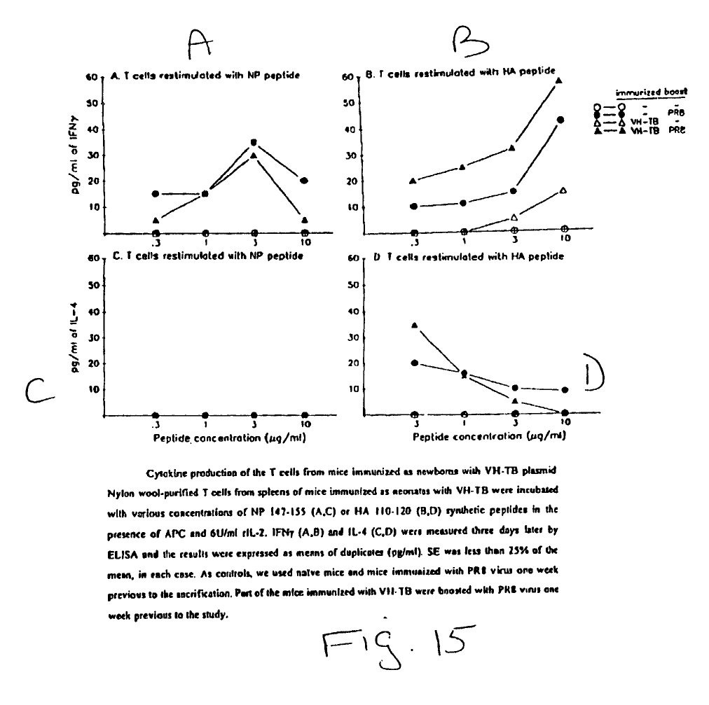

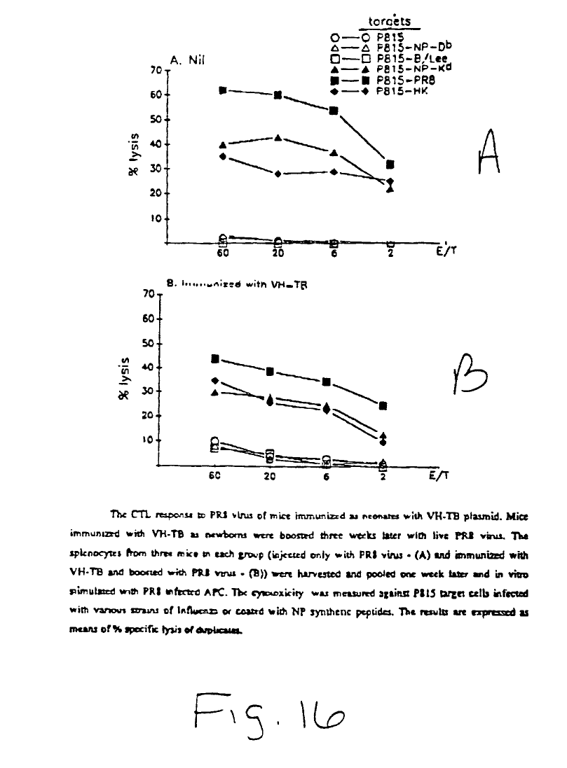

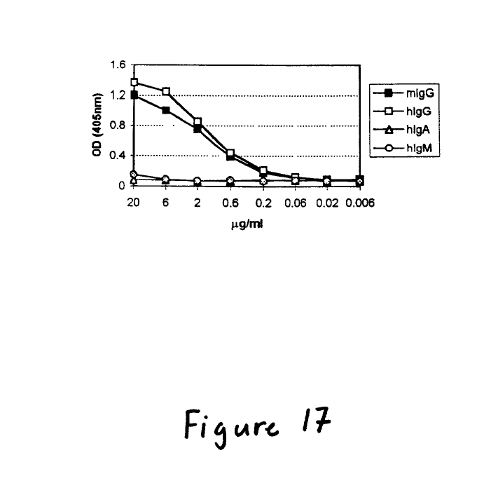

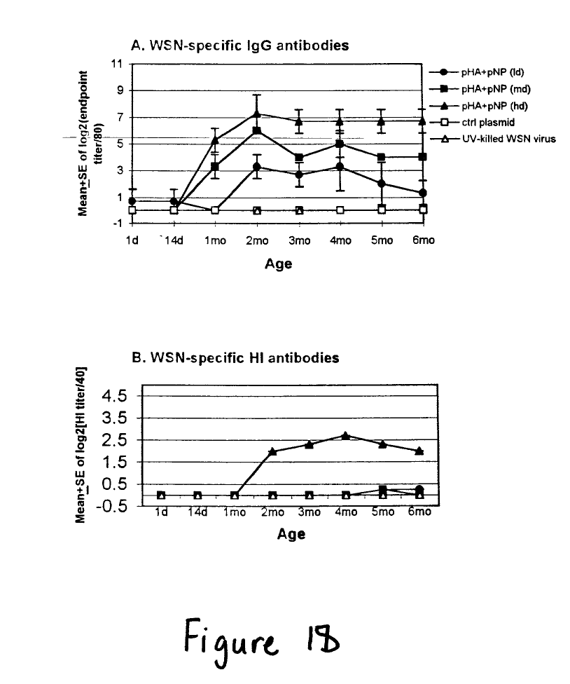

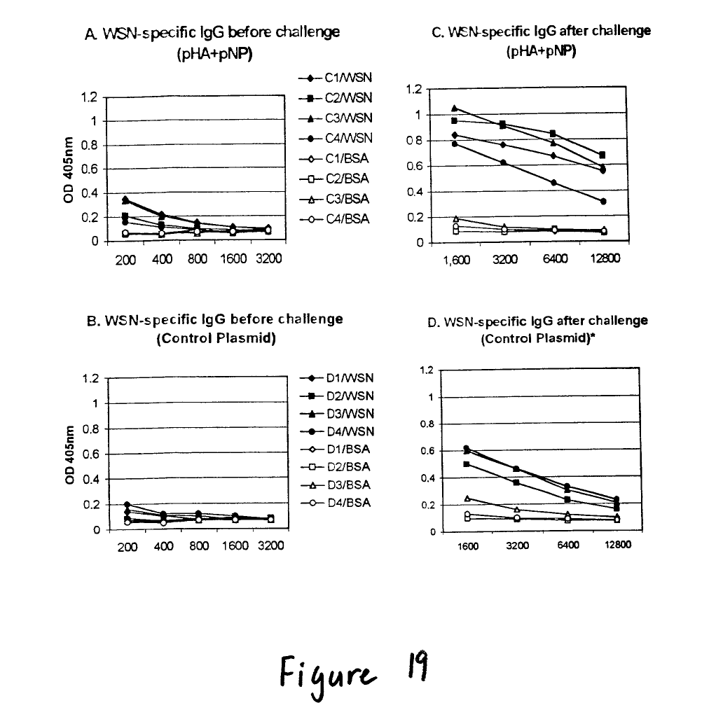

Induction of cross-reactive CTLs via DNA immunization. The induction of cross-reactive CTLs against NP-subtypes in adult animals immunized with type A influenza virus is well-characterized and understood to be related to the limited genetic variation of NP compared to hemagglutinin (HA) and neuraminidase (NA), which are viral surface proteins. In a similar manner, CTLs derived from newborn mice immunized with NPV1 and boosted with PR8 virus exhibited increased lysis of P815 cells infected with a variety of influenza strains, including PR8(H₁N₁), A/HK/68(H₃N₂) and A/Japan(H₂N₂) viruses, but not the Type B virus B/Lee, after in vitro stimulation with PR8 virus infected cells or NP₁₄₇₋₁₅₅ peptide (FIGS. 4A-C).

Effect of DNA immunization on pulmonary virus titer. The increased activity of CTLs in those animals vaccinated with NPV1 is correlated with decreased viral titers in lung tissue measured after aerosol challenge with one LD₁₀₀ of PR8 or HK viruses. Although no difference in viral titers was observed in mice immunized with NPV1 or control plasmid three days after PR8 challenge, a statistically significant reduction was observed in both newborn (p<0.05) and adult mice (p<0.025; Table 1) seven days after challenge. No virus was detected in the lungs of mice that survived challenge for more than 16 days. It is important to note that decreased viral titers in lung tissue were observed in mice challenged with PR8 virus one or three months after completing the immunization (p<0.05).

Effect of DNA immunization on clinical course of infection and survival. Genetic immunization of adult mice with NPV1 induced protective immunity in 80% of animals challenged with PR8 virus one month after the last immunization (p<0.01; FIGS. 5A-D) and in 57 percent of adult animals challenged three months after the last immunization (p<0.05; FIG. 5E). An increased survival after challenge was observed in three month old mice immunized with NPV1 as newborns, indicating that during the three month period a more vigorous expansion of CTL precursors was elicited after genetic immunization (p<0.02, FIG. 5B). Only 10% of adult animals challenged with HK virus survived (FIGS. 5A-D), findings that differ from those previously reported (Ulmer et al., 1993, Science 259:1745-1749) even though the DNA immunogen and Hk strain used in challenge were identical. The relative decrease in survival we observed could be explained by the intranasal route of challenge used previously (Id.), which is less likely to provide productive infection of the lower as well as upper respiratory tract compared to the aerosol challenge employed in these studies. Despite their immunoresponsiveness, one-month old mice immunized with NPV1 as newborns exhibited reduced survival after challenge with PR8 and no survival after challenge with HK virus compared to immunized adults and three month old mice infected with NPV1 as newborns, which exhibited significant survival after challenge with LD100 of PR8 virus.

The pneumonia that occurs after influenza infection is accompanied by weight loss in these animals. Adult mice treated with control plasmid and challenged with a lethal dose of PR8 gradually lost weight until they expired (Days 7-9), while the surviving animals immunized with NPV1 recovered their prechallenge body weight by Day 10 after significant initial weight loss post-challenge (Day 2-7; FIGS. 6A-B). Similar results were obtained with one-month old mice which had been immunized after birth as newborns with NPV1 (FIGS. 6A-B) or with three month old mice.

Effect of DNA immunization with a plasmid which encodes HA of influenza virus (pHA plasmid). Immunization of newborn mice with pHA according to the same protocol as NPV1 was followed by specific antibody production as early as 1 month after birth which persisted at least three months after birth (Table 2). These antibodies displayed hemagglutination inhibiting properties, like antibodies obtained by live-virus or plasmid immunization of adult mice. In consequence, immunization of neonates with pHA elicited protective, virus-specific antibodies.

Immunization of mice with pHA primed T helper cells which were then able to secrete cytokines upon in vitro restimulation with virus (Table 3). Whereas pHA injection of adult mice elicited predominantly TH1 type cells, innoculation of neonates with the same plasmid lead to the development of a mixed Th1/Th2 response. DNA immunization of neonates as well as adult mice with pHA conferred significant protection to lethal challenge (LD₁₀₀) with WSN or PR8 virus as early as one month after immunization (FIGS. 7A-D).

### Discussion

Numerous studies have indicated that the genetic immunization of adult mice, chickens, ferrets and monkeys with cDNAs containing NP or HA sequences of various strains of type A influenza virus can induce protective cellular and humoral immunity (Ulmer et al., 1993, Science 258:1745-1749; Montgomery et al., 1993, DNA and Cell Biol. 12:777-783; Fyneu et al., 1993, Proc. Natl. Acad. Sci. U.S.A. 90:11478-11482; Justevicz et al., 1995, J. Virol. 19:7712-7717; Donnely et al., 1995, Nature Med. 1:583-587). The results presented herein are the first evidence that such immunization has a comparable effect in newborn animals, and that cellular immunity is generated consequent to a strong priming effect characterized by a significant increase in the frequency of antigen-specific CTL precursors. The survival after challenge, the reduction in viral lung titers and recovery of prechallenge body weight compared to controls in animals that were vaccinated with NPV1 or pHA is indicative of effective secondary immune responses.

Previous studies in adult mice have indicated that immunization with homologous virus affords 100% protection to lethal challenge, while only 50-60% protection occurs in normal mice infused with NP-specific T cell clones (Taylor et al., 1986, Immunology 58:417-420) or in PR8-immunized B cell deficient (J_{H}D−/−) animals (Bot et al., 1996, J. Virol.. 70:5668-5672), indicating that effective protection requires both humoral and cellular responsiveness, the former presumably mitigating the spread of virus and the extent of pulmonary lesions. The absence of a protective antibody response in the studies carried out with NPV1 plasmid as well as slow expansion of CTL precursors during the first month of life may explain the relatively poor survival of one month old mice that were immunized with NPV1 plasmid as newborns. The increased survival of three month old mice immunized as newborns with NPV1 plasmid suggests that the expansion of CTL precursors continues after neonatal immunization, enabling the mice to develop a stronger cellular response when they become adults.

Further data indicates that the plasmid expressing the HA gene of WSN virus, injected after birth, elicits both humoral and cellular responses mirrored in an increased survival. For example, neonatal immunization with pHA triggered an antibody response associated with a helper response which conferred significant protection upon later challenge with influenza virus.

TABLE 1
EFFECT OF IMMUNIZATION WITH NPV1 PLASMID
ON PULMONARY VIRUS TITER MEASURED AFTER
CHALLENGE WITH LETHAL DOSES OF PR8 OR HK VIRUS

| | | challenge with | | | | | |
|---|---|---|---|---|---|---|---|
| | | 1.5 × 10⁴ | | | challenge with | | |
| age of | | TCID₅₀ PR8 virus | | | TCID₅₀ HK virus | | |
| animals | immunization | 3 d | 7 d | 16 d | 3 d | 7 d | 16 d |
| adult | nil | 4.6 ± 0.5 | 3.8 ± 0.1 | +³ | 6.4 ± 0.7 | 5.7 ± 0.3 | + |
| | PR8 virus | 0 | 0 | ND | 5.7 ± 0.3 | 0 | ND |
| | control plasmid | 4.8 ± 0.1 | 3.7 ± 0.5 | + | 6.8 ± 0.1 | 5.7 | + |
| | NPV1-1 month¹ | 4.0 ± 0.3 | 0.9 ± 1.5 | 0⁴ | 5.8 ± 0.1 | 0.6 ± 1.1 | 0 |
| | NPV1-3 months² | 4.8 ± 0.1 | 0.2 ± 0.2 | 0 | 6.9 ± 0.7 | 4.6 ± 0.8 | 0 |
| newborn | control plasmid | 5.9 ± 0 | 4.6 ± 0.2 | + | ND | ND | ND |
| | NPV1-1 month | 4.5 ± 1.2 | 1.2 ± 2.1 | 0 | 6.6 ± 0.3 | 5.1 ± 0.6 | + |
| | NPV1-3 months | 4.1 ± 0.5 | 0.9 ± 1.2 | 0 | ND | ND | ND |

Mice were sacrificed 1 month after the last immunization. Data are expressed as log₁₀ of viral titer in TCID₅₀ units.
ND-not done
¹-mice challenged 1 month after completing the immunization
²-mice challenged 3 months after completing the immunization
³-no survivors at day 16 after challenge.
⁴-pulmonary virus titer in mice which survived more than 16 days

TABLE 2
HI TITER OF BALB/C MICE IMMUNIZED WITH WSN VIRUS OR PLASMIDS

| Mice | | | Prebleeding | | | | | No. of | | | Titer 7 days after | |
|---|---|---|---|---|---|---|---|---|---|---|---|---|
| immunized | Immunization | No. of | Titer | | Time of | Titer against: | | respondents | | | boost against: | |
| as: | with | mice: | WSN | PR8 | bleeding: | WSN | PR8 | WSN | PR8 | Boost: | WSN | PR8 |
| Adults | WSN | 5 | 0^{a} | 0 | 1 mo. | 8.2 ± 1.1^{b} | 1.2 ± 0.8 | 5/5 | 5/5 | WSN | 8.2 ± 1.3 | 2.2 ± 1.6 |
| | CP | 3 | 0 | 0 | 1 mo. | 0 | 1.0 ± 0.7 | 0/3 | 1/3 | — | 0 | 0 |
| | CP | 3 | 0 | 0 | 1 mo. | 0 | 0 | 0/3 | 0/3 | WSN | 7.3 ± 5.3 | 1.3 ± 2.3 |
| | pHA | 16 | 0 | 0 | 1 mo. | 5.5 ± 3.4 | 0 | 12/16 | 0/16 | WSN | 8.3 ± 1.5 | 1.0 ± 1.9 |
| | pHA | 8 | 0 | 0 | 3 mo. | 8.7 ± 3.8 | 0 | 5/8 | 0/8 | WSN | 8.3 ± 1.5 | 2.0 ± 2.0 |
| | pHA | 9 | 0 | 0 | 6 mo. | 1.0 ± 0 | 0 | 2/9 | 0/9 | WSN | 8.3 ± 0.6 | 1.3 ± 0.6 |
| | pHA | 3 | 0 | 0 | 9 mo. | 0 | 0 | 0/3 | 0/3 | WSN | 5.6 ± 0.6 | 5.0 ± 1.7 |
| Newborns | CP | 5 | ND | ND | 1 mo. | 0 | 0 | 0/5 | 0/5 | WSN | 7.0 ± 0.8 | 0 |
| | pHA | 19 | ND | ND | 1 mo. | 5.2 ± 2.7 | 0 | 12/19 | 0/19 | WSN | 9.4 ± 0.9 | |
| | pHA | 4 | ND | ND | 3 mo. | 3.3 ± 1.5 | 0 | 3/4 | 0/4 | WSN | 2.0 ± 1.6 | |
| | | | | | | | | | | | 8.8 ± 2.9 | |
| | | | | | | | | | | | 3.2 ± 2.5 | |

^{a}0 = <1:40
^{b}log₂ dilution
ND-not done

TABLE 3
Lymphokine production by T cells from mice immunized with pHA
plasmid or WSN virus:

| Group | | | Adult mice | | Newborn mice | |
|---|---|---|---|---|---|---|
| Immunization | Boost | Lymphokines | nil* | WSN* | nil | WSN |
| nil | — | IFNγ | 0 | 0 | ND | ND |
| | — | IL-4 | 0 | 0 | ND | ND |
| CP | — | IFNγ | 0 | 11 ± 5** | 14 ± 5 | 22 ± 3 |
| | — | IL-4 | 0 | 0 | 0 | 0 |
| | WSN | IFNγ | 24 ± 1 | 158 ± 4 | 89 ± 28 | 261 ± 26 |
| | WSN | IL-4 | 236 ± 11 | 79 ± 19 | 198 ± 5 | 141 ± 39 |
| pHA | — | IFNγ | 9 ± 1 | 60 ± 2 | 0 | 29 ± 18 |
| | — | IL-4 | 0 | 0 | 2 + 2 | 6 ± 3 |
| | WSN | IFNγ | 19 ± 3 | 284 ± 10 | 38 ± 8 | 179 ± 50 |
| | WSN | IL-4 | 54 ± 3 | 31 ± 4 | 138 ± 4 | 257 ± 24 |
| WSN | — | IFNγ | 52 ± 2 | 214 ± 11 | 103 ± 30 | 51 ± 8 |
| | — | IL-4 | 48 ± 3 | 181 ± 3 | 132 ± 6 | 248 ± 20 |
| | WSN | IFNγ | 10 ± 1 | 127 ± 3 | 9 ± 5 | 61 ± 12 |
| | WSN | IL-4 | 218 ± 4 | 235 ± 12 | 228 ± 8 | 594 ± 5 |

*1.5 × 10⁵ nylon wool non-adherent splenocytes were incubated for four days with 1.5 × 10⁵ irradiated BALB/c splenocytes with or without 10 μg/ml UV-innactivated WSN virus, in presence of 1 U/ml exogenous IL-2.
**concentration of cytokines in supernatant was determined by ELISA and expressed as pg/ml. Values below background ± 3 × SD were considered 0.

### EXAMPLE

### Neonatal Immunization with a Mixture of Plasmids Expressing HA and NP Influenza Virus Antigens

The experiments described above showed that neonatal immunization of BALB/c mice with plasmids expressing NP or HA of Influenza virus is followed by priming of B, Th and CTL rather than tolerance. However, protection in terms of survival against lethal challenge with homologous or heterologous strains was not complete. Further, in the case of NP expressing plasmid, the protective immunity required a longer time to develop following neonatal inoculation, as compared to adult immunization.

In order to improve the protection conferred by plasmid vaccines, we coinjected pHA together with pNP in newborn and adult mice as a so-called “cocktail”. Each of these plasmids, which together encode the entire HA and NP proteins, produce antigens comprising Th, B and CTL epitopes. We challenged the mice at the age of 5 weeks with LD₁₀₀ of WSN virus or the drift variant, PR8 virus.

CTL and Th induced by neonatal inoculation of pHA+pNP or UV-attenuated WSN virus. The cytotoxic immunity and the cytokine profile of T cells from mice immunized as neonates with pHA+pNP or from mice immunized with UV-attenuated WSN virus was studied. FIG. 8 depicts the CTL response of mice immunized as newborns (infants) with either (A) UV-attenuated WSN virus or (B) a combination of pHA and pNP plasmids. Splenocytes pooled from three mice in each group were in vitro stimulated with PR8-virus infected APC and tested against P815 cells coated with NP peptides or infected with various influenza viruses at E/T ratio of 10:1. The results are expressed as means of percent specific lysis plus or minus the standard deviation of triplicates. Splenocytes from mice immunized as neonates with UV inactivated virus did not exhibit cytotoxicity against a panel of type A Influenza viruses or against the dominant NP K^{d} epitope, following in vitro stimulation with PR8 infected APC (FIG. 8A). In contrast, neonatal immunization with pHA+pNP primed a significant cytotoxic response against H1N1 strains like PR8 and WSN, against HK that is an H3N2 strain and against the dominant CTL epitope, NP 147-155 (FIG. 8B). No response was detected against a type B virus or a peptide that binds to D^{b} instead of K^{d} class I molecules.

The T helper profile was assessed following separation of CD4⁺ T cells from 5 week-old mice immunized as neonates with pHA+pNP, UV-attenuated WSN virus or non-immunized. The CD4⁺ T cells were in vitro restimulated with a panel of sucrose-purified UV-attenuated viruses in the presence of exogenous IL-2 that greatly increased the signal over noise ratio. T cells were incubated for four days in the presence of sucrose-purified UV-inactivated viruses (3 μg/ml), APC, and rIL-2 (6 U/ml). The concentration of IFNγ and IL-4 was estimated by ELISA and the results were expressed as means of duplicates plus or minus the standard deviation (pg/ml). CD4⁺ T cells from mice immunized as newborns with pHA+pNP secreted significant amounts of IFNγ but no IL-4 when restimulated with PR8 or WSN viruses (FIG. 9A). Interestingly, CD4⁺ T cells from mice immunized as newborns with UV-attenuated WSN virus secreted besides IFNγ, significant amounts of IL-4 following restimulation with PR8 or WSN virus. In fact, even in the absence of specific antigen, the IL-2 added to the culture media was sufficient to trigger significant production of IL-4 by CD4⁺ T cells from mice immunized as neonates with UV-attenuated WSN virus. In contrast, CD4⁺ T cells from non-immunized, age matched mice did not secrete significant amounts of either IFNγ or IL-4 (FIGS. 9B and 9C).

Thus, neonatal immunization with pHA+pNP induces virus specific cross-reactive reactive CTLs and Th1 cells. In contrast, neonatal immunization with UV-attenuated WSN virus does not prime CTLs but induces Th cells that secrete IL-4 and IFNγ.

Humoral response of mice immunized as neonates with pHA+pNP. In order to estimate the titer of protective antibodies generated by neonatal immunization with virus or plasmids expressing Influenza HA and NP, we measured the hemagglutination inhibiting ability of sera harvested from 5 week-old mice. As shown in Table 4, neonatal immunization with pHA+pNP induced in 5 out of 8 mice small but significant HI titers to the homologous virus. In contrast, neonatal injection with UV-attenuated WSN virus did not prime a protective humoral response. Furthermore, studies carried out in our laboratory showed that neonatal exposure to UV-attenuated WSN virus induced long-lasting B cell unresponsiveness. Thus, neonatal unresponsiveness to the neutralizing B cell epitopes of WSN virus was due to the induction of tolerance. As further detailed, we could not test the responsiveness of newborn mice to live WSN virus, because of its lethality. In sharp contrast, live virus immunization of adult mice with WSN virus induced high titers of HI antibodies against the homologous virus. Immunization of adult mice with UV-attenuated virus or pHA+pNP induced smaller HI titers against the homologous virus (Table 4). In all cases, the HI titers against the drift variant namely PR8 virus, were not significant.

TABLE 4
HEMAGGLUTINATION-INHIBITION TITERS OF SERA
FROM MICE IMMUNIZED AS NEONATES WITH pHA + pNP

| Age of | Immunized | Number of | HI titer of antibodies against^{a} | |
|---|---|---|---|---|
| immunization | with | Mice | WSN | PR8 |
| Adult | Nil | 2 | 0^{b} | 0 |
| | UV-WSN | 3 | 4.7 ± 0.6 | 0 |
| | live WSN | 3 | 7.0 ± 1.0 | 0 |
| | pHA + pNP | 3 | 3.3 ± 1.1 | 0 |
| Neonatal | Nil | 2 | 0 | 0 |
| | UV-WSN | 3 | 0 | 0 |
| | pHA + pNP | 5^{c} | 2.2 ± 0.8 | 0 |

^{a}Results were expressed as means of log₂ individual HI titers ± SE.
^{b}Titers less than 1/40 were considered 0.
^{c}Results shown for the five responder mice out of the eight mice tested.

Thus, neonatal immunization with pHA+pNP induced suboptimal but significant titers of HI antibodies in a subset of animals. In contrast, neonatal inoculation with UV-attenuated WSN virus was not effective in inducing detectable titers of protective antibodies.

Enhanced protection against lethal challenge with Influenza virus by neonatal inoculation with pHA+pNP. FIG. 10 shows the protection against lethal challenge with WSN (A, C) or PR8 (B, D) virus of mice immunized as newborns (A, B) or adults (C, D) with a combination of pHA and pNP plasmids. As controls, we used naive mice, mice inoculated with a control plasmid (pRc/CMV) and mice immunized with pHA or pNP, separately. The mice were challenged with lethal doses of virus at four weeks following the completion of immunization. Newborn mice immunized with a dose of 25 μg+25 μg of pHA+pNP/inoculation and subsequently challenged with WSN virus displayed 100% survival, in spite of the fact that mice immunized only with pHA showed more than 50% mortality, or that mice immunized with pNP did not survive (FIG. 10A). Newborn and adult mice injected with control plasmid or non-immunized, displayed no survival when challenged with either WSN or PR8 virus, four weeks after the completion of immunization. Similarly, neonates immunized with the mixture of pHA and pNP displayed approximately 80% survival following lethal challenge with PR8 virus, compared to mice immunized with pHA or pNP alone, that showed approximately 25% and 15% survival, respectively (FIG. 10B). Adult mice immunized with both pHA and pNP were significantly more protected against WSN virus than adult mice immunized with either pHA or pNP alone (FIG. 10C). In contrast, mice immunized as adults with pNP+pHA displayed similar survival rates as compared to those immunized with pHA or pNP alone, following lethal challenge with PR8 virus (FIG. 10D).

Together, these survival data show that coinjection of plasmids expressing HA and NP of Influenza virus type A into newborn mice greatly enhanced the protection against lethal infection with two distinct strains. This is more consistent with a synergistic rather than an additive relationship between HA and NP, due to the distinct nature of the immune effectors generated by the two components of the vaccine. These results were not only in contrast to the conventional view that newborn animals do not mount an immune response to vaccines, but also were surprising in that the synergistic effect was unexpected. The data indicate that combination vaccines according to the invention may be useful in creating a broader scope of protection to a pathogen, such as, for example, to encompass strain variations or genetic drift.

Dose dependency of protection following neonatal immunization with naked DNA. Further experiments were carried out in order to estimate the dose requirements for significant protection following neonatal immunization with plasmids expressing HA and NP of Influenza virus type A. Different groups of mice were inoculated with various doses of pHA, pNP or pHA+pNP. Control groups were inoculated with CP, representing the plasmid pRc/CMV lacking Influenza virus inserts. Four weeks after the completion of immunization, the mice were challenged with LD₁₀₀ of WSN virus. The number of mice that survived the challenge was recorded (Table 5) and the recovery of the surviving mice was demonstrated by the lack of pulmonary virus 16 days after the challenge. The mice were inoculated three times with plasmid. Administration of 25 μg of pHA together with 25 μg of pNP/dose resulted in complete protection, whereas inoculation of 50 μg of pHA or pNP was followed by approximately 50% and no protection, respectively (Table 5). In order to rule out the possibility of high zone tolerance in neonates, we immunized newborn mice with decreasing doses of pHA or pNP, separately. As shown in Table 5, the percentage of surviving mice decreased in the case of pHA and did not increase in the case of pNP. In contrast, adult or neonatal immunization with doses as small as 7.5 μg of each plasmid/dose was still followed by statistically significant protection after lethal challenge with WSN virus. Immunization of neonates with similar quantities of either pHA or pNP (15 μg/dose) induced no significant protection, further underlining the tremendous beneficial effect of associating the two plasmids in the same vaccine formulation.

TABLE 5
ENHANCED PROTECTION CONFERRED BY NEONATAL OR
ADULT IMMUNIZATION WITH A COMBINATION
OF HA AND NP EXPRESSING PLASMIDS

| Age of | | | | | Percentage | |
|---|---|---|---|---|---|---|
| immu- | Quantity (μg)/dose^{a} | | | No. survivors/ | survival | |
| nization | pHA | pNP | CP | total infected | (%)^{b} | p value^{c} |
| Adult | — | — | — | 0/17 | 0 | — |
| | — | — | 50 | 0/7 | 0 | >0.1 |
| | 50 | — | — | 4/7 | 57 | 0.0003 |
| | — | 50 | — | 0/4 | 0 | >0.1 |
| | 25 | 25 | — | 5/5 | 100 | <0.0001 |
| | 15 | 15 | — | 6/6 | 100 | <0.0001 |
| | 7.5 | 7.5 | — | 6/7 | 86 | 0.0002 |
| Newborn | — | — | — | 0/10 | 0 | — |
| | — | — | 50 | 0/7 | 0 | >0.1 |
| | 50 | — | — | 5/12 | 42 | 0.01 |
| | 30 | — | — | 2/7 | 29 | >0.1 |
| | 15 | — | — | 1/6 | 17 | >0.1 |
| | — | 50 | — | 0/9 | 0 | >0.1 |
| | — | 15 | — | 0/4 | 0 | >0.1 |
| | 25 | 25 | — | 10/10 | 100 | <0.0001 |
| | 15 | 15 | | 5/6 | 83 | 0.0026 |
| | 7.5 | 7.5 | | 4/7 | 57 | 0.029 |
| | 3 | 3 | | 1/4 | 25 | >0.1 |

^{a}Mice were inoculated three times and challenged with WSN virus at 4 weeks after the completion of immunization.
^{b}Survival was followed until day 20 after the challenge.
^{c}Statistical significance of survival as compared to the nil group was estimated by Fisher's exact test.

We studied the relationship between the number of inoculations and the protection conferred by neonatal immunization with pHA+pNP. Newborn mice were inoculated at day 1, 1 and 3, or 1, 3 and 6 with a mixture of pHA and pNP plasmids. At four weeks after the completion of immunization, the mice were challenged with a lethal dose of WSN virus. As shown in FIG. 11, one or two inoculations with 25 μg of each plasmid/dose, failed to induce significant protection. Even single inoculation of a larger dose of pHA together with pNP, did not result in significant protection. Thus, distribution of the naked DNA vaccine into multiple inoculations has beneficial effects in terms of protection.

Lack of protection by neonatal immunization with UV-inactivated WSN virus. The observation described above, that live virus immunization of adult mice with WSN virus induced complete protection against homologous and heterologous challenge, correlated with the priming of a broad T and B cell response specific for the homologous strain as well as cross-reactive epitopes. We could not test the ability of WSN live-virus to induce protective immunity when inoculated in newborn mice since the injection of this neurovirulent strain of Influenza virus into neonates was lethal at doses between less than 1 μg to 25 μg of sucrose purified virus. Invariably, the injection of WSN live virus in the gluteal region of 1 day old BALB/c mice was followed by impaired thriving beginning with 24-48 hours after inoculation and culminating with dehydration and death at 3 to 5 days postinjection (FIG. 12). Distinct batches of WSN virus displayed less pronounced but significant and reproducible lethality in terms of percentage survivors. Consequently, we carried out further experiments with UV-attenuated WSN virus, that is similar to the conventional killed Influenza virus vaccine. In sharp contrast to the adult mice immunized with UV-attenuated WSN virus, the neonates although surviving the immunization, were not protected against the challenge (four weeks later) with LD₁₀₀ of WSN virus (FIG. 13). This is consistent with the lack of CTL response, the deviated Th response and the B cell tolerance following neonatal inoculation of UV attenuated WSN virus, as shown above.

Clearance of the pulmonary virus in mice immunized as newborns with pHA+pNP. Immunization of adult mice with live WSN virus leads to generation of optimal titers of protective antibodies specific for the homologous strain (Table 4). A subsequent exposure to the same strain of virus does not lead to infection due to the presence of hemagglutination inhibiting antibodies, that prevent the virus binding to the sialoreceptors on the epithelial cells of the respiratory tract. Indeed, no pulmonary virus could be detected as early as three days after homologous challenge of mice immunized with live WSN virus (Table 6). In contrast, non-immunized mice or mice injected with CP as adults or neonates displayed significant pulmonary virus titers at day 3 and 7 after infection. All of the mice immunized with pHA+pNP as adults or newborns, although displaying significant pulmonary virus at day 3, showed no virus at day 7 following infection with WSN virus (Table 6). Furthermore, the mice immunized with pHA successfully cleared the virus by day 7. However, not all the mice immunized with pHA survived the challenge (FIG. 10), probably because of the extensive DTH reaction due to delayed clearance of the virus. Together, these data suggest that, while the plasmid immunization did not induce optimal titers of neutralizing antibodies capable to prevent the homologous infection, the T cell memory response led to effective clearance of the virus, in mice immunized either as adults or as neonates with pHA+pNP.

TABLE 6
CLEARANCE OF THE PULMONARY VIRUS BY MICE
IMMUNIZED AS NEONATES OR ADULTS WITH A
COMBINATION OF PLASMIDS EXPRESSING HA AND NP

| Age of | Log₁₀ of TCID₅₀ (mean ± SE)^{a} | | |
|---|---|---|---|
| immunization | day 3 | day 7 | day 20 |
| Adult mice | | | |
| injected with: | | | |
| Nil | 5.4 ± 0.7 | 3.7 ± 0.3 | +^{b} |
| CP | 4.9 ± 0.5 | 2.8 ± 0.5 | |
| WSN virus | <1.0^{c} | <1.0 | <1.0 |
| NPV1 | 4.8 ± 0.1 | † | |
| pHA | 2.0 ± 2.2 | 1.4 ± 0.8 | <1.0 |
| NPV1 + pHA | 4.4 ± 1.1 | <1.0 | <1.0 |
| Newborn mice | | | |
| injected with: | | | |
| CP | 4.2 ± 0.5 | † | † |
| NPV1 | 4.7 | † | † |
| pHA | 4.0 ± 0.6 | <1.0 | <1.0 |
| NPV1 + pHA | 3.4 ± 1.2 | <1.0 | <1.0 |

^{a}At day 3 and 7 after the lethal challenge with WSN virus, the pulmonary virus titers were estimated. At day 20, all the surviving mice were sacrificed and the lung titers measured.
^{b}No surviving mice.
^{c}Titers were considered lower than 1 if infectious virus was not detected.

Conclusion. In contrast to neonatal inoculation of UV-attenuated WSN virus, which is similar to the conventional killed vaccine (that fails to trigger a protective immune response), we show that neonatal coadministration of two plasmids expressing NP (pNP) and HA (pHA) induces protection against lethal challenge with the homologous virus and a drift variant.

Whereas HA bears dominant B and Th epitopes that are mostly strain or subtype specific, NP carries major cross-reactive CTL epitopes. Neonatal inoculation of pHA+pNP was followed by induction of CTLs that displayed cross-reactivity against various type A strains (FIG. 8). Furthermore, neonatal DNA immunization induced CD4⁺Th1 cells specific for epitopes shared by WSN virus and the drift variant, PR8 virus (FIG. 9). Finally, DNA immunization of newborn mice elicited protective antibodies against the homologous strain of virus, that was used for cloning the HA insert from pHA (Table 4). However, only 5 out of 8 mice were responders and the HI titers were significantly reduced as compared to adult mice immunized with live or UV-attenuated virus. Together, the virus-specific CTL, Th and B cells mediated a significantly increased protection against lethal challenge with WSN, in mice immunized as neonates or as adults (FIG. 10). In the case of the drift variant PR8 virus, the enhanced protection was due to the induction of PR8 specific Th and CTL, since no PR8 specific HI antibodies were measured (Table 4). The dose-protection relationship shown in Table 5, suggests strong synergism between the main immune effectors since lower doses of pHA+pNP were sufficient to induce levels of protection that could not be obtained with either pHA or pNP. In particular, although pNP elicited CTL against the major epitope NP 147-155 shared by PR8 and WSN virus, it failed to induce significant protection in terms of survival against the lethal challenge with WSN virus. The most reasonable explanation is the enhanced virulence associated with increased replication of the WSN strain due to a mutation in neuraminidase, so that CTL alone are not sufficient for significant protection against this particular strain.

In stark contrast with neonatal immunization with pHA+pNP, inoculation of WSN virus was not followed by protection. First, injection of live WSN virus in newborn mice was lethal (FIG. 12). Since inoculation of live WSN virus in adult mice was not lethal and induced complete protection against homologous challenge, this result supports the concern that live viral vaccines may induce serious side effects due to the immaturity of the neonatal immune system. Secondly, neonatal inoculation with UV-attenuated WSN virus, although not lethal because of the impairment of virus replication, did not elicit protection (FIG. 13). No CTL or B cells secreting protective antibodies were primed by UV-attenuated virus inoculated into newborn mice (FIG. 8 and Table 4). Whereas the lack of cytotoxicity may be easily explained by the lack of synthesis of viral proteins, the absence of an humoral response is most probably due to the immaturity of the neonatal immune system since adults mounted HI antibodies to UV-attenuated WSN virus. Indeed, recent data suggest that neonatal exposure to UV-attenuated WSN virus induces B cell tolerance. Further, neonatal inoculation with UV-attenuated virus induced CD4⁺ Th cells that secreted IFNγ and IL-4 (FIG. 10). It is not clear at this point how much of the IL-4 is due to the Th cells specific for culture media proteins, although we used for immunization virus purified by sucrose-gradient ultracentrifugation. It is noteworthy to mention that immunization of adult mice with UV-attenuated WSN virus, in contrast to neonatal immunization, resulted in significant but not complete protection to homologous challenge (FIG. 13). Thus, neonatal and adult immunization with UV-attenuated Influenza virus, that is similar to the conventional vaccine, appears to be less effective as compared to DNA immunization with mixtures of plasmids encoding multiple Influenza antigens.

In conclusion, neonatal inoculation of plasmids expressing HA and NP of Influenza virus was followed by priming of CTL, Th and B cells as well as increased protection against lethal challenge with two strains of virus. The data indicate that, rather than having a tolerizing effect, T cell immunity and humoral immunity are induced by neonatal DNA inoculation. In contrast, neonatal immunization with UV-attenuated WSN virus (analogous to a conventional vaccine) did not induce protection and live-virus inoculation of newborn mice was lethal.

### EXAMPLE

### Immune Responsiveness Following Neonatal Inoculation with a Plasmid Expressing an Ig Chimera Bearing T and B Epitopes of Hemagglutinin

It has been shown that self immunoglobulin molecules are effective vehicles for delivering foreign epitopes to MHC class-II molecules in the endosomal compartment of professional APC. We have engineered a chimeric gene by replacing the CDR3 and CDR2 segments of the VH fragment from an anti-arsonate mAb with the gene segments encoding major HA epitopes: HA 110-120 that is recognized by CD4⁺ T cells in the context of I-E^{d} class-II molecules and HA 150-159 respectively, that is a B cell epitope. Subsequently, the VH-TB chimeric gene was inserted into a mammalian expression vector bearing the CMV initial-early promoter and the BGH poly-adenylation signal. Further studies showed that myoblast cells transfected with the VH-TB plasmid secrete the chimeric protein in the supernatant.

Recent studies showed that neonatal inoculation with plasmids expressing the circumsporozoite antigen of Plasmodium yoelii induced tolerance to major epitopes previously defined in adults but not to non-dominant epitopes (Mor et al. 1996, J. Clin. Invest. 98:2700). We used the VH-TB chimera that bears defined T and B cell Influenza virus epitopes to inoculate neonatal mice and tested the priming effect of the VH-TB plasmid subsequent to the inoculation in adult or newborn mice.

The immune response generated by adult immunization with VH-TB plasmid. Adult BALB/c mice immunized with VH-TB plasmid develop both T and B cell immunity (Table 7). The CD4⁺ T cells separated from adult mice immunized with VH-TB at day 7 after the completion of immunization, secreted significant amounts of IFNγ but no IL-4 when restimulated with PR8 virus or a construct bearing the HA 110-120 peptide. In contrast, CD4⁺ T cells from PR8 immunized mice secreted both IFNγ and IL-4. Adult mice immunized with VH-TB mounted HA 150-159 specific antibodies at 4 weeks following the completion of immunization (Table 7). The titers of HA 150-159 and PR8-specific antibodies measured in VH-TB immunized adult mice were significantly lower than those of the mice immunized with live PR8 virus. Thus, VH-TB immunization of adult mice induced immune responses to the Th as well as the B cell epitope encoded by the chimeric gene.

TABLE 7
THE IMMUNE RESPONSE OF ADULT MICE TO VH-TB PLASMID

| | Cytokine production by CD4⁺ T cells | | | | | | | | | |
|---|---|---|---|---|---|---|---|---|---|---|
| | In vitro stimulation with^{a}: | | | | | | | | Antibody response^{b} | |
| Mice immunized | Nil | | IgG2b | | IgG-gal-HA | | PR8 virus | | anti- | anti-HA |
| with: | IFNγ | IL-4 | IFNγ | IL-4 | IFNγ | IL-4 | IFNγ | IL-4 | PR8 | 150-159 |
| Nil | 0 | 0 | 0 | 0 | 0 | 0 | 0 | 0 | 0 | 0 |
| B/Lee/40 virus | 0 | 0 | 0 | 0 | 0 | 0 | 0 | 0 | 0 | 0 |
| PR/8/34 virus | 0 | 0 | ND^{c} | ND | ND | ND | 74 ± 3 | 24 ± 3 | 42 ± 9 | 12 ± 4 |
| VH-TB | 0 | 0 | 0 | 0 | 39 ± 6 | 0 | 56 ± 3 | 0 | 4 ± 2 | 5 ± 3 |
| plasmid | | | | | | | | | | |

^{a}Negatively selected CD4⁺ T cells were restimulated for four days in the presence of 5 μg/ml of antigen. The concentration of cytokines in the supernatant was determined by ELISA and expressed as mean ± SD of duplicates in pg/ml.
^{b}The binding of antibodies to PR8 or HA 150-159 coupled to BSA was estimated by sandwich RIA using 1/100 dilutions of sera and iodinated rat anti-mouse k light chain antibodies. The standard curve was constructed using B2H1 HA-specific antibodies. Results were expressed as mean ± SD of triplicates (μg/ml).
^{c}ND - not done.

Cellular responsiveness subsequent to the neonatal inoculation of VH-TB plasmid. We separated CD4⁺ T cells from 4 week-old mice immunized as neonates with VH-TB and we tested their proliferation upon in vitro stimulation with HA 110-120 peptide or NP 147-155 peptide. Negatively selected CD4+ cells from mice immunized with VH-TB as neonates were incubated with APC in the presence of various concentrations of Np 147-155 or HA 110-120 synthetic peptides. Tritiated thymidine was added after 72 hours and the radioactivity incorporated was measured after another 14 hours. The results are expressed as means of triplicates, plus or minus the standard deviation of proliferation indices. Some mice immunized with VH-TB were boosted with PR8 virus. As controls, we used naive age-matched mice and mice immunized with live PR8 virus one week prior to sacrifice. Some of the mice were boosted with live PR8 virus at the age of 3 weeks, in order to address the question of tolerance induction. As shown in FIG. 14B, the CD4⁺ T cells from mice immunized as neonates with VH-TB and boosted with PR8 virus proliferated to a similar extent as the CD4⁺ T cells from mice immunized with live-virus at the age of 3 weeks. In contrast, the CD4+T cells from non-immunized mice or mice immunized as newborns with VH-TB did not proliferate when restimulated with HA 110-120 peptide. No significant proliferation was measured when the CD4⁺ T cells where stimulated with NP 147-155 peptide, that is a major H-2 K^{d} epitope (FIG. 14A).

We tested the ability of nylon-wool purified T cells to produce cytokines following in vitro stimulation with NP 147-155 or HA 110-120 peptide (FIG. 15). Specifically, nylon wool purified T cells from spleens of mice immunized as neonates with VH-TB were incubated with various concentrations of NP 147-155 (A, C) or HA 110-120 (B, D) synthetic peptides in the presence of APC and 6 U/ml rIL-2. IFNγ (A, B) and IL-4 (C<D) were measured three days later by ELISA and the results were expressed as means of duplicates (pg/ml). SE was less than 25% of the mean in each case. As controls, we used naive mice and mice immunized with PR8 virus one week prior to sacrifice. Part of the mice immunized with VH-TB were boosted with PR8 virus one week before the study. The T cells from mice that received an inoculation with live PR8 virus, previously immunized or not with VH-TB, secreted significant IFNy but no IL-4 when restimulated with NP 147-155 peptide (FIGS. 15A, C). Furthermore, significant amounts of IFNγ and IL-4 were produced by T cells from mice injected with live-virus, that were previously immunized or not with VH-TB as neonates (FIGS. 15B, D). The T cells from mice immunized as neonates with VH-TB and not boosted with PR8 virus secreted low but measurable amounts of IFNγ when in vitro stimulated with HA 110-120 peptide (FIG. 15B). Interestingly, the T cells from mice immunized with live-virus displayed dissimilar profiles of IFNγ and IL-4 secretion depending on the concentration of HA 110-120 peptide: whereas at lower concentrations IL-4 dominated, at higher concentrations the T cells produced more IFNγ and less IL-4.

In further experiments, mice were immunized with VH-TB as newborns and boosted three weeks later with live PR8 virus. The splenocytes from three mice in each group were harvested and pooled (see FIG. 16) one week later and in vitro stimulated with various strains of influenza or coated with NP synthetic peptides. The results are expresed as means of percent specific lysis of duplicates. The mice inoculated as neonates with VH-TB mounted significant cytotoxicity subsequent to live PR8 virus boost (FIG. 16). Splenocytes harvested from mice injected with live virus and previously immunized or not with VH-TB, after in vitro stimulation with PR8 virus, lysed the target cells infected with PR8 or HK virus, or coated with NP 147-155 peptide. Thus, neonatal inoculation with VH-TB did not impair a subsequent T cell response to the live PR8 virus.

Humoral responsiveness following the neonatal inoculation of VH-TB plasmid. Neonatal inoculation of the VH-TB plasmid was not followed by the induction of humoral responses, as revealed by the lack of PR8 neutralizing antibodies (Table 8). The binding activity for HA 150-159 peptide or PR8 virus of the sera of mice immunized as newborns with VH-TB, was similar to that of naive mice (Table 8). Neonatal injection of VH-TB plasmid did not induce unresponsiveness to PR8 virus, since mice boosted with live-virus showed unaffected neutralizing responses. Furthermore, the response to the HA 150-159 peptide, that is a major B cell epitope expressed by VH-TB, was not impaired by neonatal inoculation of the plasmid, as revealed by the ELISA data (Table 8).

TABLE 8
THE HUMORAL RESPONSIVENESS OF MICE
IMMUNIZED AS NEONATES WITH VH-TB PLASMID

| Group | | HI titer against | Binding to^{b}: | |
|---|---|---|---|---|
| Immunized | Boost | PR8 virus^{a} | HA 150-159 | PR8 virus |
| — | — | 0 | 177 ± 33 | 163 ± 20 |
| VH-TB | — | 0 | 175 ± 61 | 183 ± 17 |
| — | PR8 | 7.0 ± 1.0 | 352 ± 48 | 337 ± 79 |
| VH-TB | PR8 | 6.0 ± 0.7 | 308 ± 39 | 354 ± 26 |

^{a}Results were expressed as mean ± SE of log₂ HI titers. HI titers less than 40 were considered 0.
^{b}The binding of antibodies to the B epitope and PR8 virus was estimated by sandwich ELISA using sera at a dilution of 1/200 and biotin-conjugated goat anti-mouse IgG antibody. Results were expressed as mean ± SEM of OD₄₅₀.

Conclusion. The foregoing studies show that mice injected as newborns with VH-TB and boosted with PR8 virus developed:

1) antibodies that are neutralizing for PR8 virus and bind the HA 150-159 peptide (Table 8);

2) T cells that secreted IFNγ and IL-4 following in vitro stimulation with HA 110-120 peptide (FIG. 15);

3) CD4⁺ T cells that proliferated upon in vitro stimulation with HA 110-120 peptide (FIG. 14);

4) CTLs that lysed target cells infected with type A Influenza viruses or coated with NP 147-155 peptide that is not carried by VH-TB (FIG. 16).

These results demonstrate that neonatal inoculation of VH-TB may enhance Th or CTL responses in the case of subsequent exposure to live virus. Although neonates responded less effectively than the adults to VH-TB, the fact that they mounted an immune response remains surprising in view of conventional notions of the tolerizing, rather than immunizing, effect of neonatal vaccination. Accordingly, this example demonstrates that the DNA vaccine compositions of the invention may be used with conventional vaccination procedures to provide an enhanced immune response comprising both T-cell and humoral components.

### EXAMPLE

### Neonatal Immunization in Baboons

The experiments described below tested the ability of protype DNA vaccine against influenza virus to prime lasting immunity when administered to newborn non-human primates. The results indicate that neonatal DNA vaccination triggers virus-specific and neutralizing antibodies of titers and persistence depending on the vaccine dose. No anti-dsDNA antibodies were induced. The dominant subtype was 1gGl, similar to that elicited by virus immunization at older age. Subsequent exposure to influenza virus, more than one year after immunization, revealed significantly increased recall responses of the baboons vaccinated with DNA during the neonatal stage. Both the systemic and local humoral responses, as well as the peripheral T cell immunity, were enhanced in the baboons primed with DNA vaccine as neonates. Thus, neonatal DNA vaccination of non-human primates triggered immune memory that persisted beyond infancy.

### Materials and Methods

### Animal and Vaccination

Baboons (Papio) were housed and bred according to government regulations, at the Regional Primate Center of the University of Oklahoma Health Sciences Center.

The newborn baboons were inoculated with plasmids pHA and pNP (21) expressing the hemagglutinin (HA) of A/32/WSN HIM and the nucleoprotein (NP) of A/34/PR/8 HINT] influenza virus on day 1, 14 and 28 after birth (Table 9).

TABLE 9
SCHEDULE OF VACCINATION AND INSTILLATION
WITH INFLUENZA VIRUS OF BABOONS

| Group designation/ | | | Schedule of | Age at instillation |
|---|---|---|---|---|
| animal number | Vaccine | Dose/inoculation | vaccination | with live virus |
| A (1-4) | pHA + pNP | 40 μg + 40 μg | d1, 14, 28^{a} | ND^{b} |
| B (1-4) | pHA + pNP | 200 μg + 200 μg | d1, 14, 28 | ND |
| C (1-4) | pHA + pNP | 1 mg + 1 mg | d1, 14, 28 | C1 18 mo |
| | | | | C2 17 mo |
| | | | | C3 15 mo |
| | | | | C4 14 mo |
| D (1-4) | CP | 2 mg | d1, 14, 28 | D1 18 mo |
| | | | | D2 17 mo |
| | | | | D3 15 mo |
| | | | | D4 13 mo |
| E (1-3) | UV-inactivated | 50 μg | d1 | ND |
| | WSN virus | | | |

^{a}Days after birth.
^{b}Not done.

Different doses of plasmids (40, 200 or 1000 pg/plasmid/dose, corresponding to group A, B and C) and controls (2 mg of control plasmid CP—group D or 50 μg of inactivated influenza virus on day 1—group E) were included. The plasmid mixture was dissolved in 0.5 ml of sterile PBS and administered bilaterally, in the quadriceps muscle (0.25 ml/site) by injection. The number of DNA vaccinated baboons in each group was 4.

### Viruses and Administration of Influenza Virus

The WSN virus was grown on Madine Darby bovine kidney carcinoma (MDBK) cells with DMEM supplemented with 0.45% BSA, at 37° C. under humidified atmosphere and in the presence of 5% CO₂. At 48 hours, the supernatant was harvested and stored at −70° C. The viruses A/HK/68 (H3N2), A/Jap/57 (H2N2), A/PR/8/34 (HIN1) and the H1 reassortant P50 strain were grown on 10 day embryonated chicken eggs as previously described (Kilbourne, E. D. et al., 1968, J. Virol. 2:281-286; Palmer, D. F. et al., 1975, Immunol. Ser. 6:51-58). The viability and titer of viruses was assessed by multiplication on Madine Darby canine kidney carcinoma (MDCK) cells and standard hemagglutination of chicken red blood cells (Animal Technologies, Tyler—Tex.).

In order to obtain purified virus for ELISA assay, supernatant containing live WSN virus was centrifuged on 30% sucrose gradient for 90′ at 112,500 g. The pellet was resuspended in sterile PBS and the virus titer assessed by hemagglutination and MDCK assay. In certain assays, UV-killed WSN virus was used. Briefly, sucrose-purified virus was exposed for 15 minutes to short-wave UV light (UVP Inc., San Gabriel—Calif.) in a Petri dish placed on ice, under constant stirring. The viability of virus was monitored using MDCK assays.

For administration of WSN influenza virus, the vaccinated baboons (age at treatment given in Table 9) were sedated with ketamine and placed in dorsal decubit. Intratracheal intubation was performed using a soft 8 French by 15 pediatric feeding tube (Becton Dickinson) that was inserted through the glottis into the trachea, using a 6 French intubating stilet. The tracheal tube was taped in place and 1 ml of sterile saline containing 10⁸ TCID₅₀ WSN virus titrated on MDCK cells, was slowly administered using a syringe attached to an 8 French by 15-inch catheter inserted 1 cm past the end of the tracheal tube. An additional volume of 2 ml of sterile PBS was instilled in order to clear the dead volume. After the infection, the animals were returned to their normal habitat and they were daily observed for any change of clinical status or behavior.

### Sample Harvesting and Preparation

Blood was harvested from anesthetized baboons by venipuncture of the saphena vein. The separated sera were processed before hemagglutination inhibition (HI) assay and ELISA as follows: for HI, the sera were treated overnight at 37° C. with 100 U/ml of receptor destroying enzyme (RDE—neuraminidase; Sigma, St. Louis—Mo.) for the removal of non-specific virus binding factors like serum sialoproteins, that may lead to false positive results. The RDE was removed prior to the assay by 30′ treatment at 56° C. in the presence of calcium chloride (Palmer et al., 1975, Immunol. Ser. 6:51-78).

Low titers of anti-bovine serum albumin (BSA) antibodies were noted in the sera of baboons presumably due to exposure to cow milk and/or low contamination with BSA of the influenza virus inoculum. To minimize the background binding to BSA, aliquots of sera were depleted of anti-BSA antibodies by pre-incubation for 30 minutes with BSA-coated chicken red blood cells (RBC) at 37° C. and removal of RBC by centrifugation. Prior to use in the ELISA assays, as control for binding to sucrose-purified virus grown on BSA supplemented medium, the sera was always tested for reactivity against BSA.

Nasal washes were harvested under anesthesia, by inserting a pediatric feeding tube 2 to 4 cm into one nostril of animals placed in lateral decubit. Three ml of sterile PBS were instilled in the nostril via an attached syringe and the resulting wash collected in a Petri dish was immediately stored at −70° C.

### Measurement of Antibody Titers by Elisa

An indirect ELISA assay was used to determine levels of virus-specific IgG antibodies in the sera and nasal washes of immunized animals. Plastic microwells of Nunclmmuno-plates (Nalge Nune Int., Tustin—Calif.) were coated overnight at 4° C. with 50 μl of sucrose-purified WSN virus in carbonate buffer (pH=9) at a concentration of 10 μg/ml. As control, wells were coated with 0.1% BSA (Sigma, St. Louis—Mo.). After washing with PBS—0.05% Tween 20, the wells were blocked for 1 hour at 37° C. with non-mammalian proteins (PBS with 30%-Seablock; Pierce, Rockford—Ill.), washed again and incubated with various dilutions of samples in PBS—10% Seablock, overnight at 4° C. After washing five times, the wells were incubated with polyclonal goat anti-human IgG antibody coupled with alkaline phosphatase (Sigma Immunochemieal, catalog no. A3188) diluted (1:1000) in PBS—10% Seablock supplemented with 0.05% Tween. The anti-IgG antibody was previously screened by direct ELISA for binding to purified monkey IgG (Sigma), human IgG (Sigma), baboon serum and lack of binding to purified human IgA (Dako; Carpinteria—Calif.) and IgM (Sigma) (FIG. 17). After 2 hour incubation at room temperature, the wells were extensively washed and incubated with substrate (pNPP, Sigma) until the reaction developed. For the characterization of the IgG subtypes and IgA antibodies, anti-baboon IgGI, IgG2, IgG3, IgG4 and IgA reagents were used. The absorption (OD405 nm) was read using an automated ELISA reader (ThermoMax, Molecular Devices) equipped with a specific software (SoftMax).

The ELISA assay for the detection of dsDNA-specific IgG antibodies in the sera of DNA vaccinated baboons, was carried out using a commercial kit (Sigma Diagnostics, catalog no. EIA503-A). This kit uses the same polyclonal goat anti-human IgG antibodies that were tested for binding to monkey IgG. Positive, negative controls and standards were included in the assay.

### Measurement of Antibody Titers by Hemagglutination Inhibition (HI)

First, the appropriate virus dilution was defined by hemagglutination of chicken RBC. Namely, a virus dilution that was four-fold lower than the highest dilution associated with hemagglutination was used in the HI assays, for all influenza virus strains included in the study: the HI strains WSN, PR8, P50; the H2 strain Jap and the H3 strain HK.

Various dilutions of RDE-treated sera were incubated in 96-well flexible U-bottom plates with defined amounts of live virus diluted in PBS. After 45′ incubation of virus with diluted sera, chicken RBC were added and the hemagglutination was read after 1 hour incubation at room temperature. Controls like non-immune baboons serum, as well as wells devoid of virus or serum were run simultaneously. The HI titer was read as the highest dilution of serum that inhibited hemagglutination.

### In Vitro Inhibition of Influenza Virus Replication in Permissive Cells

Serum aliquots were heated for 30′ at 56° C. to inactivate complement. Serial dilutions in sterile PBS were incubated with a defined amount of sucrose-purified WSN virus (1×10⁵ TCID₅₀ per well) for 1 hour at 37° C. Positive controls (no serum or nonimmune baboon serum) and negative controls (no virus) were run in parallel. The mixture was added to MDCK cells that had been plated one day before at a density of 2×10⁴ cells/well in 96-well flat bottom plates. Before the addition of virus-serum mixture, the MDCK cells were briefly washed with Trypsin-EDTA (GIBCO; Grand Island—N.Y.) to facilitate virus infection. After one hour incubation of permissive MDCK cells with virus serum mixtures, DMEM supplemented with 10%-FCS was added and the cells were incubated for another 48 hours at 37° C. in a humidified atmosphere with 5% CO₂. The supernatants were harvested and incubated with chicken RBC for one hour at room temperature, to assess the extent of hermagglutination (Palmer et al., 1975, Immunol. Ser. 6:51-78). The endpoint titers representing the ex vivo virus neutralization ability of sera were read as the highest dilution associated with complete inhibition of replication in permissive MDCK cells, as measured by the hemagglutination assay.

### In Vitro Inhibition of Antigen Processing and Presentation

Various dilutions of complement-depleted baboon sera were incubated with 10 μg/ml (total volume of 50 μl) UV-inactivated sucrose-purified WSN virus for 1 hour at 37° C. The resulting mixtures were added to professional antigen presenting M12 cells (mouse B cell lymphoma, I-E^{d+}) and 14-3-1 TcH (T cell hybridoma bearing an TCR specific for HA 110-120 in the context of I-E^{d+} and expressing a reporter (3-galactosidase transgene under the control of IL-2 promoter, Bot, A. et al., 1996, J. Immunol. 157:3436-3442). The occupancy of TCR by class II peptide complex is rapidly followed by transcription of the reporter gene from the IL-2 promoter. The incubation of antigen presenting cells (APC), TcH and antigen/antibody complexes was carried out in RPMI supplemented with 10%-FCS for four hours at 37° C., in 96-well flat bottom plates (ratio of APC to TcH was 2×10⁴/1×10⁴). Supernatants were then removed and the cells were fixed in the plate with 0.2%-glutaraldehyde/2%-formaldehyde for 5 minutes at 4° C. After wash with PBS, X-gal substrate (Boehringer Mannheim, Indianapolis—Ind.) was added according to a previously described procedure (Bot, A. et al., 1996, J. Immunol. 157:3436-3442). The percentage of activated TcH was estimated by microscopy after overnight incubation at 37° C. Endpoint titers were read as the highest dilution of serum associated with less than 0.5% activated TcH. The starting serum dilution was 1/16. Controls run in the absence of serum or with non-immune serum gave similar activation profiles at all dilutions, consisting in approximately 10% activated TcH. Independently, direct cellular toxicity of sera was ruled out: 4-hour incubation of APC and TcH in the presence of serum resulted in less than 3% trypan blue positive cells.

### In Vitro Assessment T Cell Response

Four months after the intratracheal challenge with influenza virus, blood was harvested by venipuncture on heparin and peripheral blood mononuclear cells (PBMC) were separated by centrifugation on Ficoll (Pharmacia Biotech, Uppsala—Sweden) gradient (30 minutes at 2,000 RPM and 20° C). The PBMC were washed twice in HL-1 medium (BioWhittaker, Walkersville—Md.) supplemented with L-Glutamine and antibiotics. Finally, the PBMC were resuspended in HL-1 medium with 50 μM mercaptoethanol.

Part of the PBMC were incubated in 96-well flat bottom plates at a concentration of 4×10⁵/150 μl of culture medium, with or without sucrose purified WSN virus. The final concentration of UV-killed or live virus was 4 μg/ml. In parallel, the cells were incubated with medium alone. The experiment was carried out in duplicates. The rest of PBMC was divided into three subsets/each baboon: one was incubated in HL-1 medium overnight (responder cells, 4×10⁵ cells/150 μl) and the other two (stimulator cells) were infected with Vacc-NP or Vacc-T7 (MOI=10) overnight, at 37° C. and 5% CO₂ atmosphere. Vacc-NP and Vacc-T7 are recombinant vaccinia viruses that express the NP of influenza virus H1N2 and the negative control T7 protein. The syngeneic stimulator cells were washed twice and were added to the stimulator cells at a ratio responder/stimulator of 5:1. The experiment was run in duplicates.

At 72 hours after the incubation, 100 μl of supernatant/well was harvested for the analysis of IFNγ by ELISA (monkey IFNγ detection kit; Biosource International, Cammarilo—Calif.). A similar amount of fresh culture medium supplemented with 7.5 U/ml of human recombinant IL-2 (Boehringer Mannheim) was added to the wells. After two additional days of in vitro culture, BrdU was added to the wells. The BrdU incorporation was assessed at 18 hours by using a detection kit, based on tagged BrdU-specific antibodies (BrdU labeling and detection kit III, Boehringer Mannheim GmbH). The data were acquired using an automated ELISA reader (ThermoMax, Molecular Devices).

### Statistical Analysis

The Wilcoxon rank-sum test using the method of small sample table with exact significance levels (Rosner, B. et al., 1995, In Fundamentals of Biostatistics (Fourth Edition). Duxbury Press pp. 551-584), was applied to compare the magnitude of secondary responses in baboons vaccinated with DNA as neonates with the controls. This statistical method was preferred to analyze HI titers and ex vivo neutralizing titers due to the nonparametric nature of the data (normal approximation not applicable).

### Results

### Generation and Kinetics of Virus Specific Antibodies by Neonatal DNA Vaccination of Baboons

Previous results indicated that vaccination of newborn baboons with pHA+pNP resulted in generation of influenza virus—specific antibodies that were detectable as early as 4 weeks after birth, i.e. at the time of the second boost (Bot, A. et al., 1999, Viral Immunol. 12:91-96). The long-term kinetics of influenza virus-specific antibodies in the sera of the baboons that had been vaccinated with DNA as infants, was assessed by ELISA and HI (FIG. 18). Persistence of circulating virus-specific IgG antibodies beyond 6 months, depended on the dose of vaccine. Thus, whereas the virus-specific antibody titers in the baboons immunized with intermediate (group B) or low (group A) doses of DNA vaccine (200 μg or 401 μg of each plasmid/dose) decayed within months, the antibody titers in the baboons immunized with the highest dose of DNA vaccine (1 mg/plasmid/dose; group C) were sustained through more than six months. The titer of virus-neutralizing antibodies, a subset of the virus-specific antibodies detectable by ELISA, was measured by hemagglutination inhibition. Only the baboons immunized with the highest dose of DNA vaccine showed detectable titers of HI antibodies, thought to represent virus-neutralizing antibodies (FIG. 18). By the age of one year and a half, three out of four baboons from group C still displayed reduced HI titers against homologous WSN virus (Table 10).

TABLE 10
Titer of hemmagglutination-inhibiting antibodies in the sera of babbons
immunized as neonates with pHA + pNP (group C) or control plasmid (group D) and
subsequently instilled with 10⁸ TCID₅₀ of WSN influenza virus. The results are
representative for two independent measurements.

| Baboon | | | Day 0^{a} | | | | | Day 14 | | | Day 134 |
|---|---|---|---|---|---|---|---|---|---|---|---|
| number | WSN | PR8 | P50 | JAP (H2) | HK (H3) | WSN | PR8 | P50 | JAP (H2) | HK (H3) | WSN |
| D1 | 0^{b} | 0 | 0 | 0 | 0 | 5,120 | 80 | 80 | 0 | 0 | 2,560 |
| D2 | 0 | 0 | 0 | 0 | 0 | 2,560 | 80 | 0 | 0 | 0 | 640 |
| D3 | 0 | 0 | 0 | 0 | 0 | 5,120 | 160 | 80 | 0 | 0 | 2,560 |
| D4 | 0 | 0 | 0 | 0 | 0 | 2,560 | 160 | 160 | 0 | 0 | 1,280 |
| Geometric | 0 | 0 | 0 | 0 | 0 | 3,620 | 113 | 80 | 0 | 0 | 1,522 |
| mean (group D) | | | | | | | | | | | |
| C1 | 0 | 0 | 0 | 0 | 0 | 10,240 | 640 | 320 | 0 | 0 | 640 |
| C2 | 80 | 0 | 0 | 0 | 0 | 40,960 | 640 | 160 | 0 | 0 | 2,560 |
| C3 | 160 | 0 | 0 | 0 | 0 | 10,240 | 160 | 80 | 0 | 0 | 1,280 |
| C4 | 160 | 0 | 0 | 0 | 0 | 5,120 | 160 | 80 | 0 | 0 | 1,280 |
| Geometric | 92 | 0 | 0 | 0 | 0 | 12,177 | 320 | 135 | 0 | 0 | 1,280 |
| mean (group C) | | | | | | | | | | | |

^{a}HI titers at various intervals after the challenge (time of challenge is “Day 0”).
^{b}Titers below 80 were expressed as “0”.

No antibody titers, as determined by ELISA or HI, were triggered by inoculation of dose-matched control plasmid (group D) or single injection at day 1 after birth, of UV-inactivated influenza virus (group E) (FIG. 18).

A potential concern associated with DNA vaccination is whether antibodies may be induced against ds-DNA, since such antibodies have been implicated in the pathogenesis of systemic lupus erythematosus (SLE). This is particularly important in view of the direct relationship between the efficiency of the vaccine in primates and the dose of plasmid (FIG. 18). Specifically, a total dose of 6 mg of DNA vaccine was required to trigger persisting titers of specific antibodies. However, measurement of anti-ds-DNA IgG antibodies in the serum of DNA vaccinated baboons showed titers that were not significantly increased over the background (<150 IU/ml), irrespective of the dose of vaccination and time-point of measurement (between 0 and 6 months after birth). Further, the baboons immunized with the highest dose of DNA vaccine failed to mount detectable titers of anti-ds-DNA antibodies after the boost with influenza virus, at the age of 1.5 years.

Thus, naked DNA vaccination of newborn non-human primates triggered antibody responses specific for the expressed antigen rather than plasmid vector. However, the strict dose-relationship of the immune response suggests that the amount of expressed antigen may be a limiting factor for the immunogenicity of neonatal DNA vaccines.

### Memory Response to Influenza Virus, of Baboons Immunized as Newborns with DNA Vaccine

To test the induction of immune memory to viral epitopes expressed by the DNA vaccine, WSN virus was administered to the baboons primed with the highest dose of DNA vaccine (group C) as well as to controls (group D). The virus was administered via the respiratory tract (10⁸ TCID₅₀/baboon, titrated on MDCK cells and corresponding to 106 mouse infectious doses) around the age of 1 year and a half.

The presence and titer of antibodies in the sera of baboons before and subsequent to viral exposure, were assessed by ELISA. The virus-specific antibody response was not mirrored by detection of infectious virus and viral antigen in the respiratory tract of baboons, or by clinical signs reminiscent of influenza. The results suggest that despite a lack of productive infection, the administration of WSN virus to baboons resulted in significant humoral responses that allowed the assessment of immune memory. Before the boost, only modest reactivity was noted, particularly in the group vaccinated with pHA+pNP. Two weeks after virus boost, we have noted significant increase of antibody titers against WSN virus in sera from baboons immunized at birth with DNA vaccine or inoculated with CP (FIG. 19). This increase of virus-specific IgG, as determined by ELISA, was more pronounced in the group vaccinated at birth with pHA+pNP as compared to CP (FIG. 19, panels C and D: titers higher than 12,800 in vaccinated versus smaller than 12,800 in control baboons).

A different method to assess antibody responses, namely HI that measures the titer of virus-neutralizing antibodies, also indicated a similar memory response. The baboons immunized as neonates with pHA+pNP displayed modest HI titers against homologous virus before the boost (only 3 out of 4 animals, Table 10). In contrast, none of the control baboons displayed detectable HI titers before exposure to WSN virus. Significant increase (1-2 logs) in the HI titers against homologous virus were noted in all the baboons instilled with WSN influenza virus. However, again, the titers against homologous virus were higher in the baboons primed as neonates with pHA+pNP as compared to the controls (the geometric mean of titers in the group C was fourfold higher; p<0.05 by Wilcoxon rank-sum test; Table 10). Four months after the challenge, however, the level of HI antibodies declined to similar levels in both the primed and non-primed baboons (Table 10). Modest HI titers were measured against influenza virus strains of similar subtype (PR8 and P50—H1N1) but no detectable titers were measured against viruses of different subtype (Jap H2N2 or HK H3N2) (Table 10). This demonstrates that the specificity of triggered antibodies was associated with non-cross reactive HA epitopes from the homologous virus. A slightly increased antibody response against variant strains was noted in the case of baboons primed with pHA+pNP (Table 10). The IgG subtype analysis showed that IgGl was the dominant component in 3-month old baboons vaccinated with pHA+pNP (Table 11).

TABLE 11
The isotype profile of virus-specific antibodies induced by neonatal DNA
vaccination of babbons.

| | Group/baboon | IgG subtypes^{a} | | | |
|---|---|---|---|---|---|
| Timing | number | IgG1 | IgG2 | IgG3 | IgG4 |
| Age of | C1 | 0.317(+) | 0.084 | 0.050 | 0.056 |
| 3 months^{b} | C2 | 0.319(+) | 0.084 | 0.066 | 0.036 |
| | C3 | 0.413(+) | 0.117 | 0.076 | 0.051 |
| | C4 | 0.308(+) | 0.101 | 0.074 | 0.039 |
| At virus | C1 | 0.172 | 0.116 | 0.128 | 0.065 |
| challenge | C2 | 0.108 | 0.070 | 0.063 | 0.054 |
| (12-18 months)^{b} | C3 | 0.167 | 0.100 | 0.107 | 0.079 |
| | C4 | 0.058 | 0.051 | 0.045 | 0.050 |
| 14 days after | C1 | 0.434(+) | 0.117 | 0.093 | 0.053 |
| virus challenge | C2 | 0.413(+) | 0.093 | 0.082 | 0.047 |
| | C3 | 0.547(+) | 0.156(+) | 0.107 | 0.056 |
| | C4 | 0.429(+) | 0.134(+) | 0.095 | 0.060 |
| | D1 | 0.297(+) | 0.060 | 0.040 | 0.049 |
| | D2 | 0.203 | 0.037 | 0.051 | 0.035 |
| | D3 | 0.221 | 0.043 | 0.036 | 0.049 |
| | D4 | 0.126 | 0.115 | 0.032 | 0.059 |

^{a}The values correspond to average optical densities of triplicate determinations after subtraction of background, using sera diluted at 1:50. The values higher than 3 × background were labeled as (+).
^{b}Baboons from group D were not included since they did not display detectabcle IgG antibodies in a pilot screening.

Subsequent virus boost, around 1 year and a half, resulted in the increase of IgGI antibodies (4 out of 4 baboons) as well as IgG2 antibodies (in 2 out of 4 baboons) in the animals primed with pHA+pNP. The control baboons showed lower titers of IgG1 antibodies after virus challenge (1 out of 4). Lower binding of WSN-specific antibodies in Table 11 (subtyping) versus the data presented in Table 10 (HI assay) and FIG. 19 (IgG ELISA), may be explained by differences in the sensitivity of the assays.

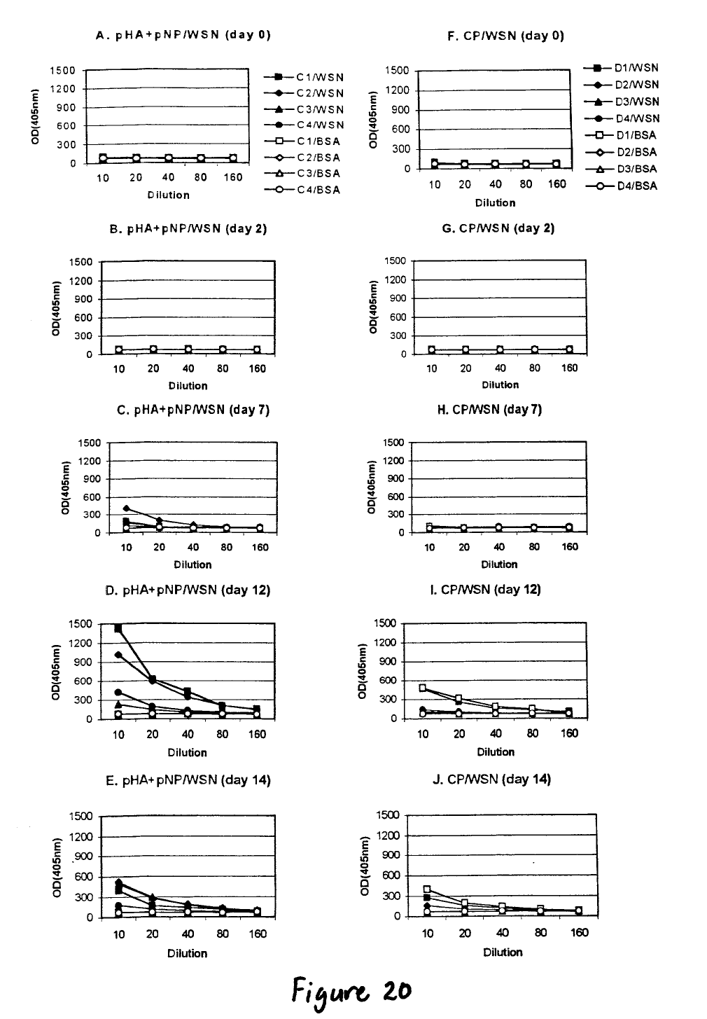

Finally, the levels of WSN-specific antibodies in the nasal washes of DNA vaccinated baboons, were determined before and at various intervals after viral boost. By ELISA assay, a transient increase of IgG antibodies specific for the homologous virus was observed in the nasal washes harvested from the baboons vaccinated with pHA+pNP as neonates and boosted one year and a half later with virus (FIG. 20). On day 12 after the virus boost, the baboons from group C displayed IgG titers in the nasal wash between 10-40. The IgG titers declined by day 14 after the administration of virus. In contrast, no specific reactivity was detected in nasal washes from control baboons that received CP as neonates (FIG. 20). Only one of the baboons from the control group D displayed modest and transient reactivity against BSA, that is a potential contaminant of the virus inoculum. In addition, we used polyclonal goat anti-human antibodies to detect specific antibodies in the nasal washes. However, no viral-specific IgA antibodies were detected.

The data indicates that systemic DNA vaccination of newborn baboons resulted in enhanced local immunity upon exposure with viral antigen at the level of respiratory tract.

### Ex Vivo Neutralizing Ability of Influenza Virus-Specific Antibodies from Vaccinated Baboons

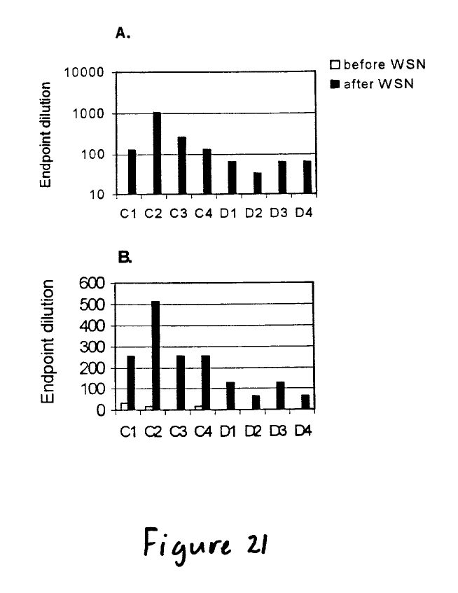

Since the epithelial cells of the respiratory tract of baboons were not permissive for WSN virus replication, the ability of virus-specific antibodies to inhibit the multiplication of virus ex vivo was assessed. The assay was designed to be able to titrate the virus-inhibitory capacity of sera from DNA vaccinated or control baboons, by preincubating defined amounts of live WSN virus with various dilutions of complement-depleted sera obtained before or 14 days after the boost. The resulting mixture was added to MDCK cells that are highly permissive for influenza virus replication. The multiplication of virus in each well was assessed by standard hemagglutination of chicken red blood cells in the presence of 48 hours MDCK culture supernatant. The higher the titer of virus-inhibiting antibodies, the higher their ability to prevent multiplication of virus up to increased dilutions of serum. The data represented as endpoint serum titers, show that after the boost, significantly higher inhibitory titers were measured in the baboons vaccinated with DNA as neonates as compared to controls (FIG. 21A). Whereas all the sera from control baboons exhibited titers between 10-100, the sera from vaccinated baboons showed titers between 100-1000, approximately one order of magnitude higher (p<0.05 by Wilcoxon rank-sum test). However, no ex vivo inhibition of virus replication in MDCK cells could be measured in sera harvested before the virus boost, correlating with low HI and ELISA titers.

It has previously been shown that protective anti-influenza virus polyclonal antibodies inhibit the processing and presentation of a dominant I-E^{d}-restricted viral epitope by mouse professional APC to specific T cell hybridoma, by facilitating an extensive degradation of the antigen in the endolysosomal compartment of APC (Bot, A. et al., 1996, J. Immunol. 157:3436-3442). Such an assay has been used to corroborate the previously described results regarding the protective ability of influenza virus-specific antibodies in baboons. After pre-incubation of defined amounts of sucrose-purified UV-inactivated virus with various dilutions of complement depleted sera, the immune complexes were added to M12 A-PC and specific TcH for 4 hours. The assay was developed and the degree of TcH activation assessed as described above and in Bot, A. et al., (1996, J. Immunol. 157:3436-3442). The higher the titer of virus-binding antibodies, the higher their ability to inhibit the presentation of the dominant I-Ed restricted HA epitope to specific TcH. As shown in the FIG. 21B, the endpoint titers corresponding to sera from baboons vaccinated with DNA as newborns and boosted with virus around the age of 1.5 years, were consistently higher than those obtained from control baboons, inoculated as neonates with CP and subsequently instilled with WSN influenza virus. Further, three of the four baboons vaccinated as neonates with pHA+pNP exhibited modest titers before the boost. Finally, the pattern of titers corresponding to the inhibition of epitope presentation (FIG. 21B), correlated with the profile of inhibition of virus multiplication in MDCK cells (FIG. 21A). Together, these data show that antibodies triggered by neonatal DNA priming followed by virus boost, are endowed with protective functions.

### Cellular Immune Response of Baboons Primed with DNA Vaccine as Newborns

The question as to whether neonatal vaccination of baboons with pHA-pNP modified the responsiveness to influenza virus, in terms of cellular immunity has been addressed. PBMC were obtained from baboons immunized with pHA+pNP (group C, highest dose) or injected with control plasmid (group D) and subsequently challenged with influenza virus via tracheal route. The PBMC were harvested at 4.5 months after the instillation with virus. At that time, the baboons were older than 1 year and a half. In parallel experiments, the PBMC were individually stimulated with killed or live homologous virus, or with syngeneic stimulator cells infected with recombinant vaccinia virus (vacc-NP) expressing the type .,/subtype HIM 1 nucleoprotein. Negative controls were simultaneously run: incubation in the absence of virus or with syngeneic cells infected with a recombinant vaccinia virus expressing the T7 protein (Vacc-T7). The production of IFN-γ was assessed at 72 hours and the proliferation on day 6 after incubation.

As shown in the Table 12, in vitro stimulation with either killed or live WSN virus triggered significant production of IFN-γ by PBMC from challenged mice, irrespective of their priming status.

TABLE 12
The cellular immune response of baboons primed as neonates with
DNA and boosted with influenza virus.

| | Response to whole virus | | | | Response to NP^{c} | |
|---|---|---|---|---|---|---|
| | Killed WSN virus | | Live WSN virus | | | |
| | Prolif- | | Prolif- | | Prolif- | |
| Baboons | eration^{a} | IFN-γ^{b} | eration | IFN-γ | eration | IFN-γ |
| C1 | 1.9 | 101 ± 3 | 1.2 | 130 ± 5 | 2.3 | — |
| C2 | — | 32 ± 3 | 1.4 | 64 ± 10 | — | 64 ± 10 |
| C3 | 1.7 | 54 ± 17 | 2.2 | 42 ± 14 | 2.1 | — |
| C4 | — | 92 ± 46 | 1.5 | 37 ± 9 | 1.2 | 65 ± 14 |
| D1 | — | 199 ± 31 | 1.3 | 505 ± 11 | — | — |
| D2 | — | 55 ± 13 | — | 35 ± 8 | — | — |
| D3 | — | 56 ± 3 | — | 206 ± 5 | — | — |
| D4 | — | 73 ± 4 | — | 137 ± 23 | —^{d} | —^{d} |

^{a}Data expressed as proliferation index relative to cell culture medium or stimulator cells infected with control Vacc-T7. Values not significantly different from background were expressed as “—”.
^{b}Data expressed as pg/ml of IFN-γ at 72 hours in cell culture supernatants. The background in the absence of virus was 11 ± 10 pg/ml. The background after stimulation with vaccinia-T7 control was 14 ± 13 pg/ml. Values less than mean + 1 SD of background were expressed as “—”.
^{c}The response was analyzed following stimulation with Vacc-NP infected syngeneic cells. In control wells, the stimulation was carried out with Vacc-T7.
^{d}D4 exhibited significant reactivity to Vacc-T7 but not Vacc-NP and was excluded from the background assessment.

There were no clear-cut differences among the animals from group C and D regarding IFN-7 production. However, when in vitro stimulated with Vacc-NP, only two animals primed with pHA+pNP as neonates exhibited significant IFN-γ production. None of the baboons injected with control plasmid and subsequently instilled with WSN virus exhibited IFN-7 production upon in vitro stimulation of PBMC with Vacc-NP infected syngeneic cells (Table 12). Furthermore, in vitro proliferation of PBMC upon antigen stimulation was clearly dependent on the priming status: only PBMC from baboons primed with pHA+pNP displayed significant proliferation (2 out of 4 upon stimulation with killed virus, 4 out of 4 with live virus and 3 out of 4 with Vacc-NP) (Table 12). In contrast, only 1 out of 4 baboons injected at birth with CP and subsequently instilled with influenza virus, exhibited a detectable proliferative response upon PBMC stimulation with live WSN virus.

Thus, although the baboons from group C and D were challenged with the same dose of live WSN virus, the priming status determined the responsiveness of PBMC to viral antigen. This is consistent with the induction of memory T cells upon neonatal DNA vaccination, that were further expanded beyond the threshold of detection by the virus boost.

Various publications are cited herein, the contents of which are incorporated by reference in their entireties.

1. A method for immunizing an infant mammal against a target antigen, comprising inoculating the mammal with an effective amount of a nucleic acid encoding a relevant epitope of the target antigen in a pharmaceutically acceptable carrier, such that a therapeutically effective amount of the relevant epitope is expressed in the infant mammal.

2. A method for inducing a cytotoxic T cell response against a pathogen in an infant mammal, comprising inoculating the mammal with an effective amount of nucleic acid encoding more than one relevant epitope of one or more target antigen associated with the pathogen in a pharmaceutically acceptable carrier, such that therapeutically effective amounts of the relevant epitopes are expressed in the infant mammal.

3. A composition of nucleic acid encoding one or more relevant epitopes of one or more target antigens, for use in the preparation of an immunogenic composition which may be used in a method of inducing a cellular immune response in an infant mammal.
